(19) United States
(12) Patent Application Publication
(10) Pub. No.: US 2025/0163475 A1
(43) Pub. Date: May 22, 2025

(54) SYSTEMS AND METHODS FOR CELL MODIFICATION

(71) Applicant: Gracell Biotechnologies (Shanghai) Co., Ltd., Shanghai (CN)

(72) Inventors: Hua ZHANG, Shanghai (CN); Huan SHI, Shanghai (CN); Lianjun SHEN, Shanghai (CN); Wei CAO, Shanghai (CN)

(21) Appl. No.: 18/724,552

(22) PCT Filed: Dec. 26, 2022
(86) PCT No.: PCT/CN2022/141874

§ 371 Date: Jun. 26, 2024

(30) Foreign Application Priority Data
Dec. 27, 2021 (WO) PCT/CN2021/141667

(51) Int. Cl. C12N 15/90; A61K 40/11; A61K 40/31; A61K 40/42; C07K 16/28; C07K 16/30; C12N 5/0783; C12N 9/22; C12N 15/11

(52) U.S. Cl. CPC C12N 15/907; A61K 40/11; A61K 40/31; A61K 40/4202; A61K 40/4211; A61K 40/4221; A61K 40/4255; A61K 40/4269; C07K 16/2803; C07K 16/2878; C07K 16/2887; C07K 16/30; C12N 5/0636; C12N 9/22; C12N 15/11; C07K 2317/24; C12N 2310/20; C12N 2510/00

(57) ABSTRACT

Provided are systems for modifying a cell. Also provided are methods for using the systems for modifying a cell.

### CROSS-REFERENCE

This application claims the benefit of PCT/CN2021/141667, filed Dec. 27, 2021, which application is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Cell based therapy such as cell transplantation or therapeutic delivery has been gaining traction. However, the cells used in cell based therapy can express cell surface markers that trigger innate immunity. For example, CAR-T cell therapy, while showing promising therapeutic efficacy, has been linked to severe inflammatory side effects in patients. Another example is cell transplantation leading to graft versus host disease (GVHD). As a result, most current clinical trials rely on autologous cells as the primary source cells for cell based therapy.

### SUMMARY

However, such reliance on autologous cell is expensive and laborious. There remains a need for systems and methods to modify cells suitable for cell based therapy, where the modified cells can possess knocked-down or knocked-out expression of cell surface molecules such as major histocompatibility complexes (MHCs) so that these cells do not trigger innate immune responses after administering to a subject in need thereof. There also remains a need for systems and methods to modify cells to express therapeutics by the modified cells.

Described herein, in some aspects, is a system comprising: a guide nucleic acid; a gene modifying moiety; and a chimeric polynucleotide, the chimeric polynucleotide comprising: at least one expression sequence; and at least one gene modifying moiety targeting fragment, wherein the chimeric polynucleotide is circular. Described herein, in some aspects, is a system comprising: a guide nucleic acid; a gene modifying moiety; and a chimeric polynucleotide, the chimeric polynucleotide comprising: at least one expression sequence; and at least one gene modifying moiety targeting fragment, wherein (i) the chimeric polynucleotide further comprises at least one covalently closed end and/or (ii) the chimeric polynucleotide is a linear DNA. In some embodiments of any one of the systems disclosed herein, the chimeric polynucleotide comprises double-stranded DNA (dsDNA) or singled-stranded DNA (ssDNA). In some embodiments of any one of the systems disclosed herein, the chimeric polynucleotide is a vector. In some embodiments of any one of the systems disclosed herein, the chimeric polynucleotide is a minicircle. In some embodiments of any one of the systems disclosed herein, the at least one covalently closed end is at 5′ end of the chimeric polynucleotide. In some embodiments of any one of the systems disclosed herein, the at least one covalently closed end is at 3′ end of the chimeric polynucleotide. In some embodiments of any one of the systems disclosed herein, the chimeric polynucleotide comprises the at least one covalently closed end 5′ end and the at least one covalently closed end at 3′ end of the chimeric polynucleotide. In some embodiments of any one of the systems disclosed herein, the chimeric polynucleotide is a circular single stranded DNA. In some embodiments of any one of the systems disclosed herein, the chimeric polynucleotide is a linear single stranded DNA. In some embodiments of any one of the systems disclosed herein, the chimeric polynucleotide is a linear single stranded DNA with modified ends. In some embodiments of any one of the systems disclosed herein, the at least one covalently closed end comprises a telomeric end. In some embodiments of any one of the systems disclosed herein, the chimeric polynucleotide comprises the at least one gene modifying moiety targeting fragment near 5′ end of the chimeric polynucleotide. In some embodiments of any one of the systems disclosed herein, the chimeric polynucleotide comprises the at least one gene modifying moiety targeting fragment near 3′ end of the chimeric polynucleotide. In some embodiments of any one of the systems disclosed herein, the chimeric polynucleotide comprises the at least one gene modifying moiety targeting fragment near 5′ end of the chimeric polynucleotide and the at least one gene modifying moiety targeting fragment near 3′ end of the chimeric polynucleotide. In some embodiments of any one of the systems disclosed herein, the guide nucleic acid comprises a nucleic acid sequence that is at least 70%, at least 75%, at least 80%, at least 85%, at least 90%, at least 95%, at least 99% or more identical to a genomic sequence in a cell. In some embodiments of any one of the systems disclosed herein, the genomic sequence comprises a genomic locus of the cell. In some embodiments of any one of the systems disclosed herein, the genomic locus comprises T-cell receptor alpha chain constant (TRAC), beta-2-microglobulin (B2M), cluster of differentiation 38 (CD38), cytokine inducible SH2 containing protein (CISH), programmed cell death protein 1 (PD-1), cluster of differentiation 70 (CD70). In some embodiments of any one of the systems disclosed herein, the genomic locus comprises TRAC or B2M. In some embodiments of any one of the systems disclosed herein, the guide nucleic acid complexes with and directs the gene modifying moiety to the genomic sequence in the cell. In some embodiments of any one of the systems disclosed herein, the at least one gene modifying moiety targeting fragment comprises between about 10 nucleotide base pairs (bps) to about 100 nucleotide bps. In some embodiments of any one of the systems disclosed herein, the at least one gene modifying moiety targeting fragment comprises a nucleic acid sequence that is at least 70%, at least 75%, at least 80%, at least 85%, at least 90%, at least 95%, at least 99% or more identical to the guide nucleic acid. In some embodiments of any one of the systems disclosed herein, the at least one gene modifying moiety targeting fragment comprises at least one mismatch, at least two mismatches, at least three mismatches, at least four mismatches, at least five mismatches, at least six mismatches, at least seven mismatches, at least eight mismatches, at least nine mismatches, or at least ten mismatches compared to the guide nucleic acid. In some embodiments of any one of the systems disclosed herein, the at least one gene modifying moiety targeting fragment complexes with the gene modifying moiety, thereby bringing the gene modifying moiety to proximity with the chimeric polynucleotide. In some embodiments of any one of the systems disclosed herein, the at least one gene modifying moiety targeting fragment, when complexed with the gene modifying moiety, does not induce enzymatic activity of the gene modifying moiety. In some embodiment of any one of the systems disclosed herein s, the gene modifying moiety comprises a Cas protein or a mRNA encoding the Cas protein. In some embodiments of any one of the systems disclosed herein, the gene modifying moiety comprises a Cas/RNP. In some embodiments of any one of the systems disclosed herein, the gene modifying moiety comprises a Cas9/RNP. In some embodiments of any one of the systems disclosed herein, the at least one expression sequence encodes a chimeric receptor. In some embodiments of any one of the systems disclosed herein, the chimeric receptor comprises an antigen binding domain binding CD1a, CD2, CD3, CD4, CD5, CD7, CD8, CD19, CD20, CD22, CD25, CD28, CD30, CD33, CD38, CD40, CD44v6, CD47, CD52, CD56, CD57, CD58, CD70, CD79a, CD79b, CD80, CD81, CD86, CD99, CD117, CD123, CD133, CD135, CD137, CD151, CD171, CD276, BAFF-R, BCMA, B7H4, CEA, CEACM6, Claudin 18.2, CLL-1, c-Met, CS-1, CTLA-4, EGFRVIII, GPC2, GPC3, GPRC5, HER2, HER3, HER4/ErbB4, HVEM, MAGE-A, MAGE3, MSLN, MUC-1, MUC-16, NY-ESO-1, OX40, PD-1, PD-L1, PD-L2, PMSA, ROR1, TCRa, TCRb, TLR7, TLR9, VEGFR-2, WT-1, or a fragment thereof. In some embodiments of any one of the systems disclosed herein, the chimeric receptor comprises a transmembrane domain. In some embodiments of any one of the systems disclosed herein, the chimeric receptor comprises a signaling domain. In some embodiments of any one of the systems disclosed herein, the cell comprises an immune cell or a stem cell. In some embodiments of any one of the systems disclosed herein, the immune cell is a lymphocyte. In some embodiments of any one of the systems disclosed herein, the lymphocyte is a B cell. In some embodiments of any one of the systems disclosed herein, the lymphocyte is a T cell. In some embodiments of any one of the systems disclosed herein, the T cell is selected from the group consisting of: cytotoxic T cell, alpha beta T cell, a gamma delta T cell, natural killer T cell, regulatory T cell, and T helper cell. In some embodiments of any one of the systems disclosed herein, the immune cell comprises an ILC. In some embodiments of any one of the systems disclosed herein, the immune cell is derived from an iPSC. In some embodiments of any one of the systems disclosed herein, the immune cell is an iPSC derived T cell. In some embodiments of any one of the systems disclosed herein, the immune cell is an iPSC derived natural killer T cell. In some embodiments of any one of the systems disclosed herein, the immune cell is an iPSC derived macrophage. In some embodiments of any one of the systems disclosed herein, the stem cell is a hematopoietic stem cell. In some embodiments of any one of the systems disclosed herein, the stem cell is an iPSC. In some embodiments of any one of the systems disclosed herein, the system described herein further comprises a polymer, the polymer comprises an overall anionic charge. In some embodiments of any one of the systems disclosed herein, the polymer comprises an anionic polynucleotide comprising poly-glutamic acid (PGA) or poly-aspartic acid (PASA).

Described herein, in some aspects, is a composition comprising the system described herein.

Described herein, in some aspects, is a cell or a cell line comprising the system described herein.

Described herein, in some aspects, is a pharmaceutical composition comprising the system described herein or the cell described herein. In some embodiments of any one of the pharmaceutical compositions disclosed herein, the pharmaceutical composition comprises a unit dose form. In some embodiments of any one of the pharmaceutical compositions disclosed herein, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments of any one of the pharmaceutical compositions disclosed herein, the pharmaceutical composition comprises at least one additional active agent. In some embodiments of any one of the pharmaceutical compositions disclosed herein, the at least one additional active agent comprises a cytokine, a growth factor, a hormone, an enzyme, a small molecule, a compound, or combinations thereof.

Described herein, in some aspects, is a kit comprising the system described herein, the cell described herein, or the pharmaceutical composition described herein; and a container.

Described herein, in some aspects, is a method comprising contacting a cell with any one of the systems disclosed herein, wherein the system knocks in the chimeric polynucleotide at a genomic sequence in the cell, thereby expressing a chimeric receptor encoded by the chimeric polynucleotide in the cell. Described herein, in some aspects, is a method comprising contacting a population of cells with the system described herein, wherein the system knocks in the chimeric polynucleotide at a genomic sequence in the population of cells, thereby expressing a chimeric receptor encoded by the chimeric polynucleotide in the population of cells. In some embodiments of any one of the methods disclosed herein, the system described herein increases knockin efficiency of the chimeric polynucleotide in the population of cells by at least 10%, at least 20%, at least 30%, at least 40%, or more compared to a Cas protein mediated knockin efficiency of the chimeric polynucleotide in absence of the at least one gene modifying moiety targeting fragment in a comparable population of cells. In some embodiments of any one of the methods disclosed herein, the system described herein increases an expression of the chimeric polynucleotide in the population of cells by at least 10%, at least 20%, at least 30%, at least 40%, or more compared to an expression of the chimeric polynucleotide in absence of the at least one gene modifying moiety targeting fragment knocked into a comparable population of cells by a Cas protein. In some embodiments of any one of the methods disclosed herein, the system described herein increases survival rate in the population of cells by at least 10%, at least 20%, at least 30%, at least 40%, or more compared to survival rate of a comparable population of cells modified by a Cas protein mediated knockin of the chimeric polynucleotide in absence of the at least one gene modifying moiety targeting fragment.

Described herein, in some aspects, is a method comprising contacting a cell with: a first system comprising any one of the systems disclosed herein, wherein the first system comprises a first guide nucleic acid complexed with a first gene modifying moiety and a first chimeric polynucleotide comprising the at least one gene modifying moiety targeting fragment; and a second system comprising any one of the systems disclosed herein, wherein the second system comprises a second guide nucleic acid complexed with a second gene modifying moiety and a second chimeric polynucleotide comprising the at least one gene modifying moiety targeting fragment, wherein the first system introduces the first chimeric polynucleotide into a first genomic sequence in the cell and the second system introduces the second chimeric polynucleotide into a second genomic sequence in the cell.

Described herein, in some aspects, is a method comprising contacting a population of cells with: a first system comprising any one of the systems disclosed herein, wherein the first system comprises a first guide nucleic acid complexed with a first gene modifying moiety and a first chimeric polynucleotide comprising the at least one gene modifying moiety targeting fragment; and a second system comprising any one of the systems disclosed herein, wherein the second system comprises a second guide nucleic acid complexed with a second gene modifying moiety and a second chimeric polynucleotide comprising the at least one gene modifying moiety targeting fragment, wherein the first system introduces the first chimeric polynucleotide into a first genomic sequence in the population of cells and the second system introduces the second chimeric polynucleotide into a second genomic sequence in the population of cells. In some embodiments of any one of the methods disclosed herein, the first system and the second system increase knockin efficiency of the first chimeric polynucleotide and the second chimeric polynucleotide in the population of cells by at least 10%, at least 20%, at least 30%, at least 40%, or more compared to a Cas protein mediated knockin efficiency of a first and a second chimeric polynucleotide in absence of the at least one gene modifying moiety targeting fragment in a comparable population of cells. In some embodiments of any one of the methods disclosed herein, the first system and the second system increase expressions of the first chimeric polynucleotide and the second chimeric polynucleotide in the population of cells by at least 10%, at least 20%, at least 30%, at least 40%, or more compared to expressions of the first and second chimeric polynucleotide in absence of the at least one gene modifying moiety targeting fragment knocked into a comparable population of cells by a Cas protein. In some embodiments of any one of the methods disclosed herein, the first system and the second system increase survival rate in the population of cells by at least 10%, at least 20%, at least 30%, at least 40%, or more compared to survival rate of a comparable population of cells modified by a Cas protein mediated knockin of a first and a second chimeric polynucleotide in absence of the at least one gene modifying moiety targeting fragment.

Described herein, in some aspects, is a method comprising contacting a cell with: a guide nucleic acid; a gene modifying moiety; and a chimeric polynucleotide, the chimeric polynucleotide comprising: at least one expression sequence; and at least one gene modifying moiety targeting fragment, wherein the chimeric polynucleotide is circular. Described herein, in some aspects, is a method comprising contacting a cell with: a guide nucleic acid; a gene modifying moiety; and a chimeric polynucleotide, the chimeric polynucleotide comprising: at least one expression sequence; at least one gene modifying moiety targeting fragment; and at least one covalently closed end. In some embodiments of any one of the methods disclosed herein, the chimeric polynucleotide comprises double-stranded DNA (dsDNA) or single-stranded DNA (ssDNA). In some embodiments of any one of the methods disclosed herein, the chimeric polynucleotide is a vector. In some embodiments of any one of the methods disclosed herein, the chimeric polynucleotide is a minicircle. In some embodiments of any one of the methods disclosed herein, the at least one covalently closed end is at 5′ end of the chimeric polynucleotide. In some embodiments of any one of the methods disclosed herein, the at least one covalently closed end is at 3′ end of the chimeric polynucleotide. In some embodiments of any one of the methods disclosed herein, the chimeric polynucleotide comprises the at least one covalently closed end 5′ end and the at least one covalently closed end at 3′ end of the chimeric polynucleotide. In some embodiments of any one of the methods disclosed herein, the at least one covalently closed end comprises a telomeric end. In some embodiments of any one of the methods disclosed herein, the chimeric polynucleotide comprises the at least one gene modifying moiety targeting fragment near 5′ end of the chimeric polynucleotide. In some embodiments of any one of the methods disclosed herein, the chimeric polynucleotide comprises the at least one gene modifying moiety targeting fragment near 3′ end of the chimeric polynucleotide. In some embodiments of any one of the methods disclosed herein, the chimeric polynucleotide comprises the at least one gene modifying moiety targeting fragment near 5′ end of the chimeric polynucleotide and the at least one gene modifying moiety targeting fragment near 3′ end of the chimeric polynucleotide. In some embodiments of any one of the methods disclosed herein, the at least one gene modifying moiety targeting fragment comprises between about 10 nucleotide base pairs (bps) to about 100 nucleotide bps. In some embodiments of any one of the methods disclosed herein, the at least one gene modifying moiety targeting fragment comprises a nucleic acid sequence that is at least 70%, at least 75%, at least 80%, at least 85%, at least 90%, at least 95%, at least 99% or more identical to the guide nucleic acid. In some embodiments of any one of the methods disclosed herein, the at least one gene modifying moiety targeting fragment comprises at least one mismatch, at least two mismatches, at least three mismatches, at least four mismatches, at least five mismatches, at least six mismatches, at least seven mismatches, at least eight mismatches, at least nine mismatches, or at least ten mismatches compared to the guide nucleic acid. In some embodiments of any one of the methods disclosed herein, the at least one gene modifying moiety targeting fragment complexes with the gene modifying moiety, thereby bringing the gene modifying moiety to proximity with the chimeric polynucleotide. In some embodiments of any one of the methods disclosed herein, the at least one gene modifying moiety targeting fragment, when complexed with the gene modifying moiety, does not induce enzymatic activity of the gene modifying moiety.

Described herein, in some aspects, is a chimeric polynucleotide comprising: at least one expression sequence; and at least one gene modifying moiety targeting fragment, wherein the chimeric polynucleotide is circular. In some embodiments of any one of the chimeric polynucleotides disclosed herein, the chimeric polynucleotide is a vector. In some embodiments of any one of the chimeric polynucleotides disclosed herein, the chimeric polynucleotide is a minicircle.

Described herein, in some aspects, is a chimeric polynucleotide comprising: at least one expression sequence; at least one gene modifying moiety targeting fragment; and at least one covalently closed end. In some embodiments of any one of the chimeric polynucleotides disclosed herein, the at least one covalently closed end is at 5′ end of the chimeric polynucleotide. In some embodiments of any one of the chimeric polynucleotides disclosed herein, the at least one covalently closed end is at 3′ end of the chimeric polynucleotide. In some embodiments of any one of the chimeric polynucleotides disclosed herein, the chimeric polynucleotide comprises the at least one covalently closed end 5′ end and the at least one covalently closed end at 3′ end of the chimeric polynucleotide. In some embodiments of any one of the chimeric polynucleotides disclosed herein, the at least one covalently closed end comprises a telomeric end. In some embodiments of any one of the chimeric polynucleotides disclosed herein, the chimeric polynucleotide comprises double-stranded DNA (dsDNA) or single stranded DNA (ssDNA). In some embodiments of any one of the chimeric polynucleotides disclosed herein, the chimeric polynucleotide comprises the at least one gene modifying moiety targeting fragment near 5′ end of the chimeric polynucleotide. In some embodiments of any one of the chimeric polynucleotides disclosed herein, the chimeric polynucleotide comprises the at least one gene modifying moiety targeting fragment near 3′ end of the chimeric polynucleotide. In some embodiments of any one of the chimeric polynucleotides disclosed herein, the chimeric polynucleotide comprises the at least one gene modifying moiety targeting fragment near 5′ end of the chimeric polynucleotide and the at least one gene modifying moiety targeting fragment near 3′ end of the chimeric polynucleotide. In some embodiments of any one of the chimeric polynucleotides disclosed herein, the at least one gene modifying moiety targeting fragment comprises between about 10 nucleotide base pairs (bps) to about 100 nucleotide bps. In some embodiments of any one of the chimeric polynucleotides disclosed herein, the at least one gene modifying moiety targeting fragment complexes with the gene modifying moiety, thereby bringing the gene modifying moiety to proximity with the chimeric polynucleotide. In some embodiments of any one of the chimeric polynucleotides disclosed herein, the at least one gene modifying moiety targeting fragment, when complexed with the gene modifying moiety, does not induce enzymatic activity of the gene modifying moiety.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 illustrates knocking in of a TCR targeting New York Esophageal Squamous Cell Carcinoma-1 (NY-ESO-1) into cells by the system described herein. The knockin was mediated by contacting the cell with a chimeric polynucleotide comprising the modifying moiety targeting fragment described herein at both 5′ end and 3′ end of the chimeric polynucleotide. The chimeric polynucleotide was not covalently closed at both 5′ end and 3′ end of the chimeric polynucleotide. The knockin yielded a population of 34.4% cells expressing the TCR knockin (TCRVβ13.1) at the TRAC locus. “Template from column” denotes chimeric polynucleotide obtained from column purification. “Template from beads” denotes chimeric polynucleotide obtained from purification from magnetic beads. One million cells (in a final volume of 20 μl) were contacted with 0.25 μg, 0.50 μg, 0.70 μg, or 1.00 μg of chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). Flow cytometry analysis showed that CD3 was knocked out in 94.9% of the cells; and 0.18% of the cells were positive for both TCRVβ13.1 and CD3.

FIG. 2 illustrates knocking in of CAR targeting CD19 or Mesothelin into cells by the system described herein. The knockin was mediated by contacting the cell with a chimeric polynucleotide comprising the modifying moiety targeting fragment described herein at both 5′ end and 3′ end of the chimeric polynucleotide. The chimeric polynucleotide was not covalently closed at both 5′ end and 3′ end of the chimeric polynucleotide. The knockin yielded: a population of 31.4% cells expressing the CD19 targeting CAR (CD-19-CAR) at the TRAC locus; a population of 33.4% cells expressing the humanized CD19 targeting CAR (HCD19-CAR) at the TRAC locus and a population of 33.8% cells expressing the Mesothelin targeting CAR (Meso-CAR-T) at the TRAC locus. One million cells (in a final volume of 20 μl) were contacted with 0.20 μg or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 3 illustrates knocking in of dual CAR (two different CARs targeting CD19 and CD20 in the same cell, denoted as GC020; or two different CARs targeting CD19 and BCMA in the same cell, denoted as GC012L) into cells at the TRAC genomic locus by the system described herein. The knockin was mediated by contacting the cell with a chimeric polynucleotide comprising the modifying moiety targeting fragment described herein at both 5′ end and 3′ end of the chimeric polynucleotide. The chimeric polynucleotide was not covalently closed at both 5′ end and 3′ end of the chimeric polynucleotide. The knockin yielded: a population of 41.1% cells expressing the dual CAR of CD19 and CD20 via the knockin at the TRAC genomic locus (with 82.7% of the cells exhibiting TRAC knockout); and a population of 22.5% cells expressing the dual CAR of CD19 via the knockin at the TRAC genomic locus (with 90.1% of the cells exhibiting TRAC knockout). One million cells (in a final volume of 20 μl) were contacted with 0.20 μg or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 4A illustrates knocking in of HLA-E into cells by the system described herein. The knockin was mediated by contacting the cell with a chimeric polynucleotide comprising the modifying moiety targeting fragment described herein at both 5′ end and 3′ end of the chimeric polynucleotide. The chimeric polynucleotide was further modified to comprise both covalently closed 5′ end and covalently closed 3′ end. The knockin mediated by chimeric polynucleotide comprising the modifying moiety targeting fragment described herein at both ends but without covalently closed 5′ end and 3′ end yielded a population of 37.7% cells expressing HLA-E (HLA-E KI). The knockin mediated by chimeric polynucleotide comprising the modifying moiety targeting fragment described herein at both ends and with covalently closed 5′ end and 3′ end yielded a population of 54.9% cells expressing HLA-E (ds-HLA-E KI). One million cells (in a final volume of 20 μl) were contacted with 0.20 μg, 0.30 μg, or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). FIG. 4B illustrates cell viability measurements showing that the cells modified with chimeric polynucleotide comprising the modifying moiety targeting fragment at both ends and with covalently closed 5′ end and 3′ end were equally or more viable than cells modified with chimeric polynucleotide comprising the modifying moiety targeting fragment described herein at both ends but without covalently closed 5′ end and 3′ end across eight days.

FIG. 5A illustrates knockin of dual CAR targeting CD19 and BCMA (GC012HL, Dual-CAR-T) into cells by the system described herein. The knockin was mediated by contacting the cell with a chimeric polynucleotide (at an amount of 0.5 μg, 1.0 μg, 1.5 μg, or 2.0 μg) comprising the modifying moiety targeting fragment at both 5′ end and 3′ end and covalently closed 5′ end and covalently closed 3′ end, 34.6% of the cells expressed the dual CAR. One million cells (in a final volume of 20 μl) were contacted with 0.50 μg, 1.0 μg, 1.5 μg, or 2.0 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). FIG. 5B illustrates cell viability of the knocked in cells after three days.

FIG. 6 illustrates knocking in of both dual CAR (Dual-CAR-T, GC012HL) and HLA-E at TRAC locus and at B2M locus respectively, yielding 21.7% of cells expressing HLA-E and 50.1% of cells expressing dual CAR. Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 7 illustrates knocking in of both dual CAR (Dual-CAR-T) and HLA-E at B2M locus and at TRAC locus respectively, yielding 31.8% of cells expressing HLA-E R. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 8 illustrates knocking in of both dual CAR (Dual-CAR-T) and HLA-E at TRAC locus and at B2M locus led to knockout of TRAC at 96.57% and knockout of B2M at 92.48%. Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 9 illustrates knocking in of the chimeric polynucleotide, where the chimeric polynucleotide was double stranded DNA (dsDNA) comprising the modifying moiety targeting fragment described herein and was: covalently closed on both 5′ and 3′ ends (telomeric ends, top), not covalently closed on both 5′ and 3′ ends (middle); and circular (minicircle, bottom). One million cells (in a final volume of 20 μl) were contacted with 0.20 μg, 0.40 μg, or 0.80 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 10 illustrates knocking in of the chimeric polynucleotide, where the chimeric polynucleotide was single stranded (either sense or antisense strand) DNA (ssDNA). On day 8, 47.2% of the cells expressed the dual CAR (GC012HL). Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

FIG. 11A illustrates knocking in of both dual CAR (targeting both CD19 and BCMA) and HLA-E into cells by the system described herein. FIG. 11B illustrates the cell killing activity of the cells with the dual CAR knocked in by the system described herein. On day 8, the cells with CAR targeting CD19 or BCMA exhibited cell killing activity for killing B cell precursor leukemia cells (Nalm6, which expressed CD19) and cancer cells (MM.1S. RPMI-8226, and JeKo-1 cells, which all expressed BCMA).

FIG. 12A-E illustrate non-viral knockin of the chimeric polynucleotide described herein for generating CAR-T cells (Dual-CAR-T targeting CD19 and BCMA). FIG. 12A illustrates modified cells that were positive for exhibiting the CAR on day 2. FIG. 12B illustrates percentage of changes of modified cells that were positive for exhibiting the knockin. FIG. 12C illustrates cells viability of the modified cells with the knockin across 3 days (left) and after being thawed from cryopreservation (right; 1.0: cell contacted with 1.0 μg of chimeric polynucleotide; KO: control T cell with only TRAC knocked out; and T: unmodified T cell).

FIG. 12D illustrates viability and percentage of changes of the modified cells that were positive for exhibiting the knockin on day 1 after being thawed from cryopreservation. NT-Ctrl: no template control. Top: modified cells exhibiting CAR targeting CD19. Bottom: modified cells exhibiting CD3. FIG. 12E illustrates cell killing activity of the modified cells (Dual-CAR-T. ZAP-CAR-T) against cells expressing CD19 (Nalm6 cells) or BCMA (JeKo-1 and MM.1S cells).

FIG. 13A illustrates knocking in of dual CAR for targeting CD19 or CD20 at a single locus of TRAC via the system described herein. 41.1% of the cells exhibited positive expression for the dual CAR for targeting CD 19 or CD20. FIG. 13B illustrates knocking in of dual CAR for targeting CD19 or BCMA at a single locus of TRAC via the system described herein. 41.1% of the cells exhibited positive expression for the dual CAR for targeting CD 19 or BCMA.

FIG. 14 illustrates knocking in of chimeric polynucleotide by the system described here, where the chimeric polynucleotide knocked in at a locus such as TRAC or B2M was over five thousand base pairs (bps). About 20% of the modified cells were positive for exhibiting the knockin. Flow cytometry study shows that 25.2% of the cells expressed CD19 CAR. 5.77% of the cells expressed both CD19 CAR and HLA-E.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Described herein, in some aspects, is a system for modifying a cell. In some embodiments, the system modifies a cell by knocking in a chimeric polynucleotide at a genomic locus of the cell, where the chimeric polynucleotide encodes a chimeric receptor. In some embodiments, the chimeric polynucleotide, instead of encoding a chimeric receptor, encodes a major histocompatibility complex (MHC) protein. The expression of the MHC protein by the modified cell can decrease triggering of innate immune response by the modified cell in a subject. In some embodiments, the system comprises a guide nucleic acid. In some embodiments, the system comprises a gene modifying moiety, where the gene modifying moiety is complexed with the guide nucleic acid. Upon complexing with the guide nucleic acid, the gene modifying moiety is directed to the genomic locus for mediating the knockin of the chimeric polynucleotide. In some embodiments, the chimeric polynucleotide comprises at least one gene modifying moiety targeting fragment, where the at least one gene modifying target fragment can be complexed with the gene modifying moiety. The complexing between the at least one gene modifying moiety targeting fragment and the gene modifying moiety allows the gene modifying moiety and the at least one gene modifying moiety targeting fragment to be in close proximity with each other. Such close proximity between the gene modifying moiety and the at least one gene modifying moiety targeting fragment can lead to increased knockin efficiency of the chimeric polynucleotide. In some embodiments, the chimeric polynucleotide is circular. In some embodiments, the circular chimeric polynucleotide is a vector. In some embodiments, the circular chimeric polynucleotide is a minicircle. In some embodiments, the chimeric polynucleotide is linear. In some embodiments, the linear chimeric polynucleotide comprises at least one covalently closed end at either 5′ or 3′ end of the chimeric polynucleotide. In some embodiments, the linear chimeric polynucleotide comprises covalently closed ends at both 5′ end and 3′ end of the chimeric polynucleotide. The chimeric polynucleotide being circular or having at least one covalently closed end can decrease the degradation of the chimeric polynucleotide (e.g., by nuclease in a cell being modified). The decreased degradation of the chimeric polynucleotide can lead to increased abundance of the chimeric polynucleotide at the genomic locus for the knockin. In some embodiments, the chimeric polynucleotide being circular or having at least one covalently closed end increases knockin efficiency of the chimeric polynucleotide.

Described herein, in some aspects, is a cell modified with a system described herein. In some embodiments, the cell modified by the system described herein can be a cell of a cell line. In some embodiments, the cell modified by the system described herein can be formulated in a composition, a pharmaceutical composition, a kit, or a combination thereof. In some embodiments, the cell modified by the system described herein, the composition, the pharmaceutical composition, the kit, or the combination thereof can be used to treat a disease or condition in a subject in need thereof. In some embodiments, the disease or condition is neoplasia or cancer.

Described herein, in some aspects, is a method for modifying a cell. In some embodiments, the method comprises contacting the cell with the system described herein. In some embodiments, the method comprises contacting the cell with any one or any combination of the components of the system described herein. In some embodiments, the method comprises contacting the cell with a first system and a second system described herein, where the first system and the second system each knocks in a chimeric polynucleotide. In such arrangement, two chimeric polyuronides (each encoding the same or different chimeric receptor) can be knocked into the same cell. In some embodiments, the method increases the knockin efficiency of a transgene (e.g., a chimeric receptor encoded by the chimeric polynucleotide) of a cell compared to a knockin efficiency of the same transgene mediated by other methods. In some embodiments, the method increases the knockin efficiency of a transgene (e.g., a chimeric receptor encoded by the chimeric polynucleotide) of a cell compared to a knockin efficiency mediated by other methods not using the chimeric polynucleotide described herein. In some embodiments, the method increases the viability (as determined by survival rate) of the cell modified by the system described compared to a viability of a cell modified by other means (e.g., other system without using the chimeric polynucleotide described herein.

### Systems

Described herein, ins some embodiments, is a system for modifying a cell described herein. In some embodiments, the system modifies the cell by knockin in a chimeric polynucleotide, where the chimeric polynucleotide comprises at least one expression sequence. In some embodiments, the at least one expression sequence encodes a transgene or a fragment thereof. In some embodiments, the transgene comprises a chimeric receptor. In some embodiments, the transgene comprises a major histone compatibility complex (MHC) protein. In some embodiments, the transgene comprises a MCH-I protein. In some embodiments, the transgene comprises a MCH-II protein. In some embodiments, the cell can be modified by at least a first system and at least a second system, where the chimeric polynucleotide of the first system and the second system each encodes a transgene. The transgene can be same or different. For example, the first system can knockin a first chimeric receptor that is the same or different from a second chimeric receptor knocked by the second system. In some embodiments, the cell can be modified by a first system, a second system, or any additional number of system, where the modified cell can then express a first transgene, a second transgene, or any additional number of transgenes. In some embodiments, the system comprises a polymer comprising an overall anionic charge. In some embodiments, the polymer can be poly-glutamic acid (PGA) or poly-aspartic acid (PASA). The inclusion of the anionic polymer can shield the excess cationic charge of other components of the system, thereby increasing the knockin efficiency of the chimeric polynucleotide.

### Guide Nucleic Acid

In some embodiments, the system comprises a guide nucleic acid. In some embodiments, the guide nucleic comprises a two separate nucleic acid molecules, which can be referred to as a double guide nucleic acid or a single nucleic acid molecule, which can be referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule. A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid. A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid. The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used. In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a PAM within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

Guide nucleic acids of the present disclosure can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence; a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors. DNA methyl transferases. DNA demethylases, histone acetyltransferases, histone deacetylases, and combinations thereof. A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleotide can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can commonly be referred to as forming the internucleoside backbone of the guide nucleic acid. The linkage or backbone of the guide nucleic acid can be a 3′ to 5′ phosphodiester linkage.

A guide nucleic acid can comprise a modified backbone and/or modified internucleoside linkages. Modified backbones can include those that retain a phosphorus atom in the backbone and those that do not have a phosphorus atom in the backbone. Suitable modified guide nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (such as a single inverted nucleoside residue in which the nucleobase is missing or has a hydroxyl group in place thereof). Various salts (e.g., potassium chloride or sodium chloride), mixed salts, and free acid forms can also be included.

A guide nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Particularly suitable are O((CH2)nO)mCH3, O(CH2)nOCH3, O(CH2)nNH2, O(CH2)nCH3, O(CH2)nONH2, and O(CH2)nON((CH2)nCH3)2, where n and m are from 1 to about 10. A sugar substituent group can be selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH2 CH2OCH3, also known as 2′-O-(2-methoxyethyl) or 2′-MOE, an alkoxyalkoxy group).

A guide nucleic acid can also include nucleobase (or “base”) modifications or substitutions. As used herein, “unmodified” or “natural” nucleobases can include the purine bases, (e.g, adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In some embodiments, the guide nucleic acid comprises a nucleic acid sequence that is at least 70%, at least 75%, at least 80%, at least 85%, at least 90%, at least 95%, at least 99% or more identical to a genomic sequence of the genomic locus of the cell, some embodiments, the guide nucleic acid comprises a nucleic acid sequence that is 100% identical to a genomic sequence of the genomic locus of the cell. In some embodiments, the guide nucleic acid comprises a nucleic acid sequence that is at least 70%, at least 75%, at least 80%, at least 85%, at least 90%, at least 95%, at least 99% or more complementary to a genomic sequence of the genomic locus of the cell, some embodiments, the guide nucleic acid comprises a nucleic acid sequence that is 100% complementary to a genomic sequence of the genomic locus of the cell. In some embodiments, genomic locus of the cell comprises a T-cell receptor alpha chain constant (TRAC) locus. In some embodiments, genomic locus of the cell comprises Beta-2-Microglobulin (B2M) locus. In some embodiments, the genomic locus can also include genomic locus of CD38, CISH, PD-1, or CD70.

### Gene Modifying Moiety

In some embodiments, the system comprises at least one gene modifying moiety. In some embodiments, the gene modifying moiety can be complexed with at least one guide nucleic acid described herein. In some embodiments, the gene modifying moiety, upon complexing with the guide nucleic acid, is directed to the genomic locus, where the gene modifying moiety cleaves the genomic locus. In some embodiments, the cleavage mediated by the gene modifying moiety leads to a double stranded break at the genomic locus of the cell. In some embodiments, cleavage mediated by the gene modifying moiety leads to a single stranded break or a nicking of the genomic locus of the cell. In some embodiments, the cleavage mediated by the gene modifying moiety can then be repaired by the endogenous repair mechanism, where, during the repair, the chimeric polynucleotide described herein is introduced or knocked in into the cleaved genomic locus of the cell. For example, the cleavage mediated by the gene modifying moiety can induce homologous-directed repair (HDR) in the cell to be modified, where the HDR inserts the chimeric polynucleotide into the cleaved genomic locus of the cell.

In some embodiments, the gene modifying moiety comprises a nucleic acid-guided nuclease. In some embodiments, the gene modifying moiety comprises a CRISPR-Cas polypeptide. In some embodiments, the gene modifying moiety can be, for example, Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or a functional fragment thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Cas6e, Cas6f, Cas7, Cas8a, Cas8al, Cas8a2, Cas8b, Cas8c, Csn1, Csx12, Cas10, Cas10d, CaslO, CaslOd, CasF, CasG, CasH, Csy1, Csy2, Csy3, Cse1 (CasA), Cse2 (CasB), Csc3 (CasE), Csc4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csx14, CsxlO, Csx16, CsaX, Csx3, Csx1, Csx15, Csf1, Csf2, Csf3, Csf4, or Cul966; any derivative thereof; any variant thereof; or any fragment thereof. In some embodiments. Cas13 can include, but are not limited to, Cas13a, Cas13b, Cas13c, and Cas 13d (e.g., CasRx). In some embodiments, the gene modifying moiety comprises a Cas/RNP. In some embodiments, the gene modifying moiety comprises a Cas9/RNP. In some embodiments, the gene modifying moiety comprises a Cas protein or a mRNA encoding the Cas protein.

Any suitable nuclease (e.g., endonuclease) can be used in as the gene modifying moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archacal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

In some embodiments, the gene modifying moiety can be complexed with the chimeric polynucleotide described herein via the at least one gene modifying moiety targeting fragment. In some embodiments, the at least one gene modifying moiety targeting fragment, upon complexing with the gene modifying moiety, allows close proximity between the chimeric polynucleotide and the gene modifying moiety. Such arrangement allows the chimeric polynucleotide to be readily inserted into the genomic locus upon cleavage of the genomic locus by the gene modifying moiety.

### Chimeric Polynucleotide

In some embodiments, the system comprises a chimeric polynucleotide. In some embodiments, the chimeric polynucleotide comprises double-stranded DNA (dsDNA). In some embodiments, the chimeric polynucleotide comprises single-stranded DNA (ssDNA). In some embodiments, the chimeric polynucleotide comprises a combination of double-stranded DNA (dsDNA) and single-stranded DNA (ssDNA). In some embodiments, the chimeric polynucleotide comprises at least one gene modifying moiety targeting fragment to be complexed with gene modifying moiety. In some embodiments, the chimeric polynucleotide comprises at least one expression sequence. In some embodiments, the at least one expression sequence encodes at least one transgene or a fragment thereof. In some embodiments, the transgene can encode any one of the chimeric receptor described herein. In some embodiments, the at least one expression sequence can be flanked by at least one homology arm, where the homology arm comprises a nucleic acid sequence that is homologous to nucleic acid sequence of the genomic locus such as TRAC locus or the B2M locus. In some embodiments, the genomic locus can also include genomic locus of CD38, CISH, PD-1, or CD70.

In some embodiments, the chimeric polynucleotide comprises about 1,000 base pairs to about 6,500 base pairs. In some embodiments, the chimeric polynucleotide comprises about 1,000 base pairs to about 1,500 base pairs, about 1,000 base pairs to about 2,000 base pairs, about 1,000 base pairs to about 2,500 base pairs, about 1,000 base pairs to about 3,000 base pairs, about 1,000 base pairs to about 3,500 base pairs, about 1,000 base pairs to about 4,000 base pairs, about 1,000 base pairs to about 4,500 base pairs, about 1,000 base pairs to about 5,000 base pairs, about 1,000 base pairs to about 5,500 base pairs, about 1,000 base pairs to about 6,000 base pairs, about 1,000 base pairs to about 6,500 base pairs, about 1,500 base pairs to about 2,000 base pairs, about 1,500 base pairs to about 2,500 base pairs, about 1,500 base pairs to about 3,000 base pairs, about 1,500 base pairs to about 3,500 base pairs, about 1,500 base pairs to about 4,000 base pairs, about 1,500 base pairs to about 4,500 base pairs, about 1,500 base pairs to about 5,000 base pairs, about 1,500 base pairs to about 5,500 base pairs, about 1,500 base pairs to about 6,000 base pairs, about 1,500 base pairs to about 6,500 base pairs, about 2,000 base pairs to about 2,500 base pairs, about 2,000 base pairs to about 3,000 base pairs, about 2,000 base pairs to about 3,500 base pairs, about 2,000 base pairs to about 4,000 base pairs, about 2,000 base pairs to about 4,500 base pairs, about 2,000 base pairs to about 5,000 base pairs, about 2,000 base pairs to about 5,500 base pairs, about 2,000 base pairs to about 6,000 base pairs, about 2,000 base pairs to about 6,500 base pairs, about 2,500 base pairs to about 3,000 base pairs, about 2,500 base pairs to about 3,500 base pairs, about 2,500 base pairs to about 4,000 base pairs, about 2,500 base pairs to about 4,500 base pairs, about 2,500 base pairs to about 5,000 base pairs, about 2,500 base pairs to about 5,500 base pairs, about 2,500 base pairs to about 6,000 base pairs, about 2,500 base pairs to about 6,500 base pairs, about 3,000 base pairs to about 3,500 base pairs, about 3,000 base pairs to about 4,000 base pairs, about 3,000 base pairs to about 4,500 base pairs, about 3,000 base pairs to about 5,000 base pairs, about 3,000 base pairs to about 5,500 base pairs, about 3,000 base pairs to about 6,000 base pairs, about 3,000 base pairs to about 6,500 base pairs, about 3,500 base pairs to about 4,000 base pairs, about 3,500 base pairs to about 4,500 base pairs, about 3,500 base pairs to about 5,000 base pairs, about 3,500 base pairs to about 5,500 base pairs, about 3,500 base pairs to about 6,000 base pairs, about 3,500 base pairs to about 6,500 base pairs, about 4,000 base pairs to about 4,500 base pairs, about 4,000 base pairs to about 5,000 base pairs, about 4,000 base pairs to about 5,500 base pairs, about 4,000 base pairs to about 6,000 base pairs, about 4,000 base pairs to about 6,500 base pairs, about 4,500 base pairs to about 5,000 base pairs, about 4,500 base pairs to about 5,500 base pairs, about 4,500 base pairs to about 6,000 base pairs, about 4,500 base pairs to about 6,500 base pairs, about 5,000 base pairs to about 5,500 base pairs, about 5,000 base pairs to about 6,000 base pairs, about 5,000 base pairs to about 6,500 base pairs, about 5,500 base pairs to about 6,000 base pairs, about 5,500 base pairs to about 6,500 base pairs, or about 6,000 base pairs to about 6,500 base pairs. In some embodiments, the chimeric polynucleotide comprises about 1,000 base pairs, about 1,500 base pairs, about 2,000 base pairs, about 2,500 base pairs, about 3,000 base pairs, about 3,500 base pairs, about 4,000 base pairs, about 4,500 base pairs, about 5,000 base pairs, about 5,500 base pairs, about 6,000 base pairs, or about 6,500 base pairs. In some embodiments, the chimeric polynucleotide comprises at least about 1,000 base pairs, about 1,500 base pairs, about 2,000 base pairs, about 2,500 base pairs, about 3,000 base pairs, about 3,500 base pairs, about 4,000 base pairs, about 4,500 base pairs, about 5,000 base pairs, about 5,500 base pairs, or about 6,000 base pairs. In some embodiments, the chimeric polynucleotide comprises at most about 1,500 base pairs, about 2,000 base pairs, about 2,500 base pairs, about 3,000 base pairs, about 3,500 base pairs, about 4,000 base pairs, about 4,500 base pairs, about 5,000 base pairs, about 5,500 base pairs, about 6,000 base pairs, or about 6,500 base pairs.

In some embodiments, the chimeric polynucleotide is circular. In some embodiments, the circular chimeric polynucleotide is a vector. In some embodiments, the circular chimeric polynucleotide is a minicircle. In some embodiments, the chimeric polynucleotide is linear. In some embodiments, the linear chimeric polynucleotide comprises at least one covalently closed end at either 5′ or 3′ end of the chimeric polynucleotide. In some embodiments, the linear chimeric polynucleotide comprises covalently closed ends at both 5′ end and 3′ end of the chimeric polynucleotide. In some embodiments, the covalently closed end comprises a telomeric end. In some embodiments, the linear chimeric polynucleotide comprises a telomeric end at 5′ end of the chimeric polynucleotide. In some embodiments, the linear chimeric polynucleotide comprises a telomeric end at 3′ end of the chimeric polynucleotide. In some embodiments, the linear chimeric polynucleotide comprises a telomeric end at both 5′ end of the chimeric polynucleotide and 3′ end of the chimeric polynucleotide. The chimeric polynucleotide being circular or having at least one covalently closed end can decrease the degradation of the chimeric polynucleotide (e.g., by nuclease in a cell being modified). The decreased degradation of the chimeric polynucleotide can lead to increased abundance of the chimeric polynucleotide at the genomic locus for the knockin. In some embodiments, the chimeric polynucleotide being circular or having at least one covalently closed end increases knockin efficiency of the chimeric polynucleotide.

In some embodiments, the at least one gene modifying moiety targeting fragment is located near the 5′ end of the linear chimeric polynucleotide. In some embodiments, the at least one gene modifying moiety targeting fragment is located at 5′ end of the linear chimeric polynucleotide. In some embodiments, the at least one gene modifying moiety targeting fragment is located at least about 10 base pairs (bps), at least about 50 base pairs (bps), at least about 100 base pairs (bps), at least about 200 base pairs (bps), at least about 500 base pairs (bps), at least about 1000 base pairs (bps), or at least about 1500 base pairs (bps), from the 5′ end of the linear chimeric polynucleotide.

In some embodiments, the at least one gene modifying moiety targeting fragment is located near the 3′ end of the linear chimeric polynucleotide. In some embodiments, the at least one gene modifying moiety targeting fragment is located at 3′ end of the linear chimeric polynucleotide. In some embodiments, the at least one gene modifying moiety targeting fragment is located at least about 10 base pairs (bps), at least about 50 base pairs (bps), at least about 100 base pairs (bps), at least about 200 base pairs (bps), at least about 500 base pairs (bps), at least about 1000 base pairs (bps), or at least about 1500 base pairs (bps), from the 3′ end of the linear chimeric polynucleotide. In some embodiments, the at least one gene modifying moiety targeting fragment can be located near both the 5′ end of the linear chimeric polynucleotide and the 3′ end of the linear chimeric polynucleotide. In some embodiments, the at least one gene modifying moiety targeting fragment is located in proximity of the expression sequence of the chimeric polynucleotide encoding the chimeric receptor. In some embodiments, the at least one gene modifying moiety targeting fragment is located in proximity of the expression sequence of the circular chimeric polynucleotide encoding the chimeric receptor. In some embodiments, the at least one gene modifying moiety targeting fragment is located at least about 10 base pairs (bps), at least about 50 base pairs (bps), at least about 100 base pairs (bps), at least about 200 base pairs (bps), at least about 500 base pairs (bps), at least about 1000 base pairs (bps), or at least about 1500 base pairs (bps), from the expression sequence of the chimeric polynucleotide.

In some embodiments, the at least one gene modifying moiety targeting fragment comprises between about 5 base pairs to about 200 base pairs. In some embodiments, the at least one gene modifying moiety targeting fragment comprises between about 5 base pairs to about 10 base pairs, about 5 base pairs to about 20 base pairs, about 5 base pairs to about 30 base pairs, about 5 base pairs to about 40 base pairs, about 5 base pairs to about 50 base pairs, about 5 base pairs to about 60 base pairs, about 5 base pairs to about 70 base pairs, about 5 base pairs to about 80 base pairs, about 5 base pairs to about 90 base pairs, about 5 base pairs to about 100 base pairs, about 5 base pairs to about 200 base pairs, about 10 base pairs to about 20 base pairs, about 10 base pairs to about 30 base pairs, about 10 base pairs to about 40 base pairs, about 10 base pairs to about 50 base pairs, about 10 base pairs to about 60 base pairs, about 10 base pairs to about 70 base pairs, about 10 base pairs to about 80 base pairs, about 10 base pairs to about 90 base pairs, about 10 base pairs to about 100 base pairs, about 10 base pairs to about 200 base pairs, about 20 base pairs to about 30 base pairs, about 20 base pairs to about 40 base pairs, about 20 base pairs to about 50 base pairs, about 20 base pairs to about 60 base pairs, about 20 base pairs to about 70 base pairs, about 20 base pairs to about 80 base pairs, about 20 base pairs to about 90 base pairs, about 20 base pairs to about 100 base pairs, about 20 base pairs to about 200 base pairs, about 30 base pairs to about 40 base pairs, about 30 base pairs to about 50 base pairs, about 30 base pairs to about 60 base pairs, about 30 base pairs to about 70 base pairs, about 30 base pairs to about 80 base pairs, about 30 base pairs to about 90 base pairs, about 30 base pairs to about 100 base pairs, about 30 base pairs to about 200 base pairs, about 40 base pairs to about 50 base pairs, about 40 base pairs to about 60 base pairs, about 40 base pairs to about 70 base pairs, about 40 base pairs to about 80 base pairs, about 40 base pairs to about 90 base pairs, about 40 base pairs to about 100 base pairs, about 40 base pairs to about 200 base pairs, about 50 base pairs to about 60 base pairs, about 50 base pairs to about 70 base pairs, about 50 base pairs to about 80 base pairs, about 50 base pairs to about 90 base pairs, about 50 base pairs to about 100 base pairs, about 50 base pairs to about 200 base pairs, about 60 base pairs to about 70 base pairs, about 60 base pairs to about 80 base pairs, about 60 base pairs to about 90 base pairs, about 60 base pairs to about 100 base pairs, about 60 base pairs to about 200 base pairs, about 70 base pairs to about 80 base pairs, about 70 base pairs to about 90 base pairs, about 70 base pairs to about 100 base pairs, about 70 base pairs to about 200 base pairs, about 80 base pairs to about 90 base pairs, about 80 base pairs to about 100 base pairs, about 80 base pairs to about 200 base pairs, about 90 base pairs to about 100 base pairs, about 90 base pairs to about 200 base pairs, or about 100 base pairs to about 200 base pairs. In some embodiments, the at least one gene modifying moiety targeting fragment comprises between about 5 base pairs, about 10 base pairs, about 20 base pairs, about 30 base pairs, about 40 base pairs, about 50 base pairs, about 60 base pairs, about 70 base pairs, about 80 base pairs, about 90 base pairs, about 100 base pairs, or about 200 base pairs. In some embodiments, the at least one gene modifying moiety targeting fragment comprises between at least about 5 base pairs, about 10 base pairs, about 20 base pairs, about 30 base pairs, about 40 base pairs, about 50 base pairs, about 60 base pairs, about 70 base pairs, about 80 base pairs, about 90 base pairs, or about 100 base pairs. In some embodiments, the at least one gene modifying moiety targeting fragment comprises between at most about 10 base pairs, about 20 base pairs, about 30 base pairs, about 40 base pairs, about 50 base pairs, about 60 base pairs, about 70 base pairs, about 80 base pairs, about 90 base pairs, about 100 base pairs, or about 200 base pairs.

In some embodiments, the at least one gene modifying moiety targeting fragment and the guide nucleic acid share a nucleic acid sequence identity between about 60% to about 100%. In some embodiments, the at least one gene modifying moiety targeting fragment and the guide nucleic acid share a nucleic acid sequence identity between about 60% to about 70%, about 60% to about 75%, about 60% to about 80%, about 60% to about 85%, about 60% to about 90%, about 60% to about 95%, about 60% to about 96%, about 60% to about 97%, about 60% to about 98%, about 60% to about 99%, about 60% to about 100%, about 70% to about 75%, about 70% to about 80%, about 70% to about 85%, about 70% to about 90%, about 70% to about 95%, about 70% to about 96%, about 70% to about 97%, about 70% to about 98%, about 70% to about 99%, about 70% to about 100%, about 75% to about 80%, about 75% to about 85%, about 75% to about 90%, about 75% to about 95%, about 75% to about 96%, about 75% to about 97%, about 75% to about 98%, about 75% to about 99%, about 75% to about 100%, about 80% to about 85%, about 80% to about 90%, about 80% to about 95%, about 80% to about 96%, about 80% to about 97%, about 80% to about 98%, about 80% to about 99%, about 80% to about 100%, about 85% to about 90%, about 85% to about 95%, about 85% to about 96%, about 85% to about 97%, about 85% to about 98%, about 85% to about 99%, about 85% to about 100%, about 90% to about 95%, about 90% to about 96%, about 90% to about 97%, about 90% to about 98%, about 90% to about 99%, about 90% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. In some embodiments, the at least one gene modifying moiety targeting fragment and the guide nucleic acid share a nucleic acid sequence identity between about 60%, about 70%, about 75%, about 80%, about 85%, about 90%, about 95%, about 96%, about 97%, about 98%, about 99%, or about 100%. In some embodiments, the at least one gene modifying moiety targeting fragment and the guide nucleic acid share a nucleic acid sequence identity between at least about 60%, about 70%, about 75%, about 80%, about 85%, about 90%, about 95%, about 96%, about 97%, about 98%, or about 99%. In some embodiments, the at least one gene modifying moiety targeting fragment and the guide nucleic acid share a nucleic acid sequence identity between at most about 70%, about 75%, about 80%, about 85%, about 90%, about 95%, about 96%, about 97%, about 98%, about 99%, or about 100%. In some embodiments, the at least one gene modifying moiety targeting fragment comprises a nucleic acid sequence that is at least 70%, at least 75%, at least 80%, at least 85%, at least 90%, at least 95%, at least 99% or more identical to the guide nucleic acid. In some embodiments, the at least one gene modifying moiety targeting fragment comprises at least one mismatch, at least two mismatches, at least three mismatches, at least four mismatches, at least five mismatches, at least six mismatches, at least seven mismatches, at least eight mismatches, at least nine mismatches, or at least ten mismatches compared to the guide nucleic acid.

In some embodiments, the at least one gene modifying moiety targeting fragment complexes with the gene modifying moiety, thereby bringing the gene modifying moiety to close proximity with the chimeric polynucleotide. In some embodiments, the at least one gene modifying moiety targeting fragment, when complexed with the gene modifying moiety, does not induce enzymatic activity of the gene modifying moiety. For example, the at least one gene modifying moiety targeting fragment can be complexed with the gene modifying moiety (e.g., in a comparable manner as the guide nucleic acid complexing with the gene modifying moiety) without inducing the cleavage event mediated by the gene modifying moiety.

In some embodiments, the chimeric polynucleotide comprises at least one expression sequence encoding a transgene. In some embodiments, the chimeric polynucleotide encodes a transgene comprising a chimeric receptor described herein. In some embodiments, the chimeric polynucleotide encodes a transgene comprising a MHC protein described herein. In some embodiments, the MHC protein includes HLA-A, HLA-E, HLA-DM, HLA-DO, HLA-DR, HLA-DQ. HLA-DP, or a combination thereof.

### Chimeric Receptors

Described herein, in some aspects, is a cell modified by the system described herein to knock in a transgene encoded by the chimeric polynucleotide. In some embodiments, the cell can be modified by a first system and a second system to knockin two separate transgenes, the two separate transgenes can be the same or different. In some embodiments, the transgene encoded by the chimeric polynucleotide is a chimeric receptor. In some aspects, the chimeric receptor can be a chimeric antigen receptor (CAR) or T cell receptor (TCR). In some embodiments, the cell modified by the system described herein can express a transgene. In some embodiments, the cell modified by the system described herein can express a chimeric receptor described herein. In some embodiments, the cell modified by the system described herein can express a first transgene and a second transgene. In some embodiments, the cell modified by the system described herein can express a first chimeric receptor and a second chimeric receptor described herein. In some embodiments, the cell modified by the system described herein can express a first transgene, a second transgene, or any additional number of transgenes. In some embodiments, the cell modified by the system described herein can express a first chimeric receptor, a second chimeric receptor, or any additional number of chimeric receptors described herein.

In some aspects, the cell provided herein comprises one or more chimeric receptors comprising CARs. The CAR can include an extracellular domain, a transmembrane domain, or an intracellular signaling domain. The extracellular domain can include a target-specific binding element (also known as an antigen binding domain). The intracellular domain can include a costimulatory signaling region and a zeta chain portion. A costimulatory signaling region refers to a portion of the CAR comprising the intracellular domain of a costimulatory molecule. Costimulatory molecules are cell surface molecules other than antigens receptors or their ligands that may be needed for an efficient response of lymphocytes to antigen. Between the extracellular domain and the transmembrane domain of the CAR, or between the cytoplasmic domain and the transmembrane domain of the CAR, there may be incorporated a spacer domain.

As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids. With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in the present disclosure may be derived from (e.g., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker. The cytoplasmic domain or otherwise the intracellular signaling domain of the CAR of the present disclosure can be responsible for activation of at least one of the normal effector functions of the immune cell in which the CAR has been placed in. The term “effector function” refers to a specialized function of a cell.

Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the present disclosure include the cytoplasmic sequences of the TCR and co-receptors that act in concert to initiate signal transduction following antigen receptor engagement, as well as any derivative or variant of these sequences and any synthetic sequence that has the same functional capability. Signals generated through the TCR alone may be insufficient for full activation of the T cell and that a secondary or co-stimulatory signal may be included. Thus, T cell activation can be the to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences can regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the present disclosure include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the present disclosure comprises a cytoplasmic signaling sequence derived from CD3-zeta. In some embodiments, the cytoplasmic domain of the CAR can be designed to comprise the CD3-zeta signaling domain by itself or combined with any other desired cytoplasmic domain(s) useful in the context of the CAR of the present disclosure. For example, the cytoplasmic domain of the CAR can comprise a CD3 zeta chain portion and a costimulatory signaling region. The costimulatory signaling region refers to a portion of the CAR comprising the intracellular domain of a costimulatory molecule.

A costimulatory molecule is a cell surface molecule other than an antigen receptor or its ligands that may be needed for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the present disclosure is, in some cases, exemplified with 4-1BB as the co-stimulatory signaling element, other costimulatory elements are within the scope of the present disclosure.

The cytoplasmic signaling sequences within the cytoplasmic signaling portion of the CAR of the present disclosure may be linked to each other in a random or specified order. Optionally, a short oligo- or polypeptide linker, preferably between 2 and 10 amino acids in length may form the linkage. A glycine-serine doublet provides a particularly suitable linker. In some embodiments, the cytoplasmic domain is designed to comprise the signaling domain of CD3-zeta and the signaling domain of CD28. In another embodiment, the cytoplasmic domain is designed to comprise the signaling domain of CD3-zeta and the signaling domain of 4-1BB. In yet another embodiment, the cytoplasmic domain is designed to comprise the signaling domain of CD3-zeta and the signaling domain of CD28 and 4-1BB.

A CAR provided herein can comprise one or more antigen binding domains. In some cases, a CAR provided herein comprises an antigen binding domain that can target both an immune cell antigen (e.g., to inhibit killing activity of a T cell or NK cell) and a disease-associated antigen (e.g., a tumor-associated antigen). For example, an antigen binding domain targeting both immune cell antigens and cancer antigens include, but not limited to, CD2, CD3, CD4, CD5, CD7, CD8, CD30, CD38, CD45, CD48, CD50, CD52, CD56, CD69, CD100, CD122, CD132, CD137, CD161, CD159a, CD159c, CD279, CD314, CD319 (CS1) and TCR.

In some cases, a CAR provided herein comprises two antigen binding domains such that one individual CAR is a bispecific CAR, targeting two different antigens. For bispecific CAR, one antigen binding domain can target immune cell antigen, and the other antigen binding domain can target disease-associated antigen. The two antigen binding domains of a bispecific CAR can have a tandem structure, a parallel structure or a loop structure. For example, a CAR can target a tumor cell marker and CD3. The CAR can have a structure as formula I: L-scFyl-I-scFv2-H-TM-C-CD3 (I), wherein each “-” is independently a linker peptide or a peptide bond; L is optionally a signaling peptide sequence; I is a flexible linker; H is optionally a hinge region; TM is a transmembrane domain; C is a costimulatory domain; CD3 is a cytoplasmic signaling sequence derived from CD3t; one of scFv1 and scFv2 is an antigen binding domain targeting a tumor cell marker, and the other one is an antigen binding domain targeting CD3. The CAR can have a structure as formula II or II′: L-VL-scFv-VH-H-TM-C-CD3 (II), L-VH-scFv-VL-H-TM-C-CD3 (II′), wherein each “-” is independently a linker peptide or a peptide bond; the elements L. H. TM. C and CD3 as described above; scFv is an antigen binding domain targeting a tumor cell marker, VH is an anti-CD3 antibody heavy chain variable region, and VL is an anti-CD3 antibody light chain variable region; or scFv is an antigen binding domain targeting CD-3. VH is an anti-tumor cell marker antibody heavy chain variable region, and VL is an anti-tumor cell marker antibody light chain variable region. In some cases, a CAR can comprise the structure of EGFRt-CD3 scFv-CD19 scFv-Hinge-TM-CD28/41BB-CD3, wherein EGFRt is a truncated EGFR, as a safety switch (e.g., inducible cell death moiety), CD3 scFv is the svFCv fragment of the heavy and light chain variable regions of the monoclonal antibody OKT3 or UCHT1 linked by a GS linker, and the CD19 scFv fragment is the heavy and light chain variable region of the monoclonal antibody linked by a GS linker. The structure of the CAR can further comprise a hinge, transmembrane regions, costimulatory signaling region of CD28 or 41BB, and/or CD3 intracellular domain. In the present disclosure, the nucleic acid construct of EGFRt-CD3 scFv-CD19 scFv-Hinge-TM-CD28/41BB-CD3 can be inserted into a vector (e.g., a lentiviral vector). The vector can be packaged in 293T cells. T cells can be sorted from PBMC, and after activation. TCR and PD-1 genes can be knocked out by CRISPR/CAS technology. T cells can then be infected with the vectors to express the CARs. The prepared CAR-T cells can be used to detect the infection efficiency and gene editing efficiency of CAR by flowcytometry.

The immune cell marker, e.g., CD3, of the above examples can be replaced with other immune cell markers such as CD7 and CD137. In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain. VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3 (IV); L3-scFv2-R-scFv1-H3-TM3-C3-CD3 (IV′), wherein each “-” is independently a linker peptide or peptide bond; L3 is an optional signal peptide sequence; scFv1 is an antigen binding domain that targets tumor cell markers; R is a rigid or flexible joint; scFv2 is an antigen binding domain (e.g., an antibody single-chain variable region sequence) that targets T cell and NK cell consensus markers; H3 is an optional hinge region; TM3 is a transmembrane domain; C3 is a costimulatory domain; CD3 is a cytoplasmic signaling sequence derived from CD3. In some cases, a CAR comprising two antigen binding domains arranged in a loop form. In some cases, the first antigen binding domain and the second antigen binding domain is arranged, from amino terminus to carboxyl terminus, as: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have the following formula VI, VI′, VI″ or VI′″ structure: L8-VL1-VH2-I-VL2-VH1-H8-TM8-C8-CD3 (VI); L8-VH1-VL2-I-VH2-VL1-H8-TM8-C8-CD3 (VI′); L8-VL2-VH1-I-VL1-VH2-H8-TM8-C8-CD3 (VI″); L8-VH2-VL1-I-VH1-VL2-H8-TM8-C8-CD3 (VI″), wherein each “-” is independently a linker peptide or peptide bond; L8 is an optional signal peptide sequence; VH1 is an anti-tumor cell marker antibody heavy chain variable region, and VL1 is an anti-tumor cell marker antibody light chain variable region; VH2 is an anti-T cell and NK cell consensus marker (such as CD7 or CD2) antibody heavy chain variable region; and VL2 is an anti-T cell and NK cell consensus marker (such as CD7 or CD2) antibody light chain variable region; I is a flexible joint; H8 is an optional hinge region; TM8 is a transmembrane domain; C8 is a costimulatory domain; CD3 is a cytoplasmic signaling sequence derived from CD3.

In some cases, a CAR comprising two antigen binding domains are arranged in a parallel form. The parallel form can comprise a full construct of a first CAR having a first antigen binding domain linked to a full construct of a second CAR having a second antigen binding domain. An example of parallel form can be tEGFR-CD19 scFv-CD28-CD3-CD3 scFv-41BB-CD3. The tEGFR shown here can function as a safety switch, which can be replaced by other safety switches as described in the present disclosure. As described herein. CD19 scFv and CD3 scFv are two examples of antigen binding domains, which may be replaced with various antigen binding domains as described in the present disclosure, CD28 can be an example of transmembrane domain and can be replaced with other transmembrane domains described herein, 41BB can be an example of co-stimulatory domain and can be replaced with other co-stimulatory domains described herein. In some cases, a linker is used to link the first CAR and the second CAR. The linker can be a cleavable linker. The cleavable linker can be self-cleaving peptide such as 2A self-cleaving peptide.

Also contemplated in the present disclosure is a nucleic acid molecule encoding a CAR or a bispecific CAR. The nucleic acid can comprise a first sequence encoding a chimeric antigen receptor (CAR), wherein the CAR can comprise a binding moiety, which binding moiety comprises (i) a first antigen binding domain, which first antigen binding domain suppresses or decreases a subject's immune response toward the cell described herein when administered into the subject linked to (ii) a second antigen binding domain capable of binding to a disease-associated antigen, and wherein each CAR of the one or more CARs can further comprise a transmembrane domain and an intracellular signaling domain. The first antigen binding domain can target an immune cell antigen selected from the group consisting of CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2F, CD279, CD314 (NKG2D), CD305, CD335 (NKP46), CD337, CD319 (CS1), TCRα, TCRβ and SLAMF7. The second antigen binding domain can target a disease-associated antigen such as CD19. Other non-limiting examples of disease-associated antigen includes BCMA, VEGFR2, CD19, CD20, CD30, CD22, CD25, CD28, CD30, CD33, CD52, CD56, CD80, CD86, CD81, CD123, cd171, CD276, B7H4, CD133, EGFR, GPC3; PMSA, CD 3, CEACAM6, c-Met, EGFRvIII, ErbB2, ErbB3 HER-2, HER3, ErbB4/HER-4, EphA2, IGFIR, GD2, O-acetyl GD2, O-acetyl GD3, GHRHR, GHR, Flt1, KDR, Flt4, CD44V6, CEA, CA125, CD151, CTLA-4, GITR, BTLA, TGFBR2, TGFBR1, IL6R, gp130, Lewis, TNFR1, TNFR2, PD1, PD-L1, PD-L2, HVEM, MAGE-A. Mesothelin, NY-ESO-1, PSMA, RANK, ROR1, TNFRSF4, CD40, CD137, TWEAK-R, LTPR, LIFRP, LRP5, MUC1, TCRa, TCRp, TLR7, TLR9, PTCH1, WT-1, Robl, Frizzled, OX40, CD79b Claudin 18.2, Folate receptor a, Folate receptor (3, GPC2, CD70, BAFF-R and Notch-1-4.

The nucleic acid molecule can further comprise a second sequence encoding an enhancer moiety, which enhancer moiety can enhance one or more activities of the CAR when expressed in a cell. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSFIR, CTAL-4, TIM-3, CCL21, CCL19, TGFR beta, receptors for the same, functional fragments thereof, functional variants thereof, and combinations thereof. The nucleic acid molecule can further comprise a second sequence encoding an inducible cell death moiety, which inducible cell death moiety, when expressed in a cell, can effect death of the cell upon contacting the inducible cell death moiety with a cell death activator. The inducible cell death moiety can be selected from the group consisting of rapaCasp9, iCasp9, HSV-TK, ACD20, mTMPK, ACD19, RQR8, and EGFRt.

The nuclei acid molecule can further comprise a third sequence flanked by the first sequence and the second sequence, wherein the third sequence can encode a cleavable linker. The cleavable linker can be a self-cleaving peptide. The nucleic acid molecule can further comprise a regulatory sequence regulating expression of the first sequence and/or the second sequence. Also contemplated in the present disclosure is a kit comprising the nucleic acid molecule described herein. In some cases, the nucleic acid encoding the CAR described herein can be delivered into an immune cell for expression of the CAR to generate an engineered cell.

### Methods for Modifying Cell

Described herein, in some aspects, is a method for modifying a cell with a system described herein. In some aspects, the cell is modified for expressing at least one of the chimeric receptors described herein. In some aspects, the cell is modified for expressing at least one of the MHC proteins described herein. In some aspects, the cell is modified by knocking in the chimeric polynucleotide described herein, where the chimeric polynucleotide encodes the chimeric receptor or the MHC protein. In some embodiments, the cell can be modified by at least a first system and at least a second system, where the chimeric polynucleotide of the first system and the second system each encodes a transgene. The transgene of the first system and the second system can be same or different. For example, the first system can knockin a first transgene comprising the chimeric receptor or the MHC protein, and the second system can knockin a second transgene comprising a chimeric receptor or a MHC protein that is different from the first chimeric receptor or the first MHC protein. In some embodiments, the cell can be modified by a first system, a second system, or any additional number of system, where the modified cell can then express a first chimeric receptor, a second chimeric receptor, or any additional number of chimeric receptors.

In some aspects, the method comprises contacting the cell described herein with the system or any component of the system or any combination of the component of the system described herein. In some aspects, the method comprises contacting the cell with the chimeric polynucleotide, where the chimeric polynucleotide, after knocked in, encodes or expresses the chimeric receptor or the MHC protein. In some aspects, the system or any component of the system or any combination of the component of the system described herein can be readily introduced into a cell, e.g., a mammalian, bacterial, yeast, or insect cell by any method in the art. For example, the system or any component of the system or any combination of the component of the system described herein can be transfected into the cell by physical, chemical, or biological means. In some embodiments, the system or any component of the system or any combination of the component of the system described herein can be delivered into the cell via physical methods such as calcium phosphate precipitation, lipofection, particle bombardment, microinjection, gene gun, electroporation, and the like.

Physical methods for introducing the system or any component of the system or any combination of the component of the system described herein into the cell can include calcium phosphate precipitation, lipofection, particle bombardment, microinjection, gene gun, electroporation, and the like. One method for the introduction of the system or any component of the system or any combination of the component of the system described herein to the cell is calcium phosphate transfection.

Chemical means for introducing the system or any component of the system or any combination of the component of the system described herein into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of non-naturally occurring polynucleotide or vector encoding the gene modifying moiety with targeted nanoparticles.

In the case where a non-viral delivery system is utilized, an example delivery vehicle is a liposome. The use of lipid formulations is contemplated for the introduction of the system or any component of the system or any combination of the component of the system described herein into a cell (in vitro, ex vivo, or in vivo). In another aspect, the system or any component of the system or any combination of the component of the system described herein can be associated with a lipid. The system or any component of the system or any combination of the component of the system described herein associated with a lipid can be encapsulated in the aqueous interior of a liposome, interspersed within the lipid bilayer of a liposome, attached to a liposome via a linking molecule that is associated with both the liposome and the gene modifying moiety or the heterologous polynucleotide encoding the gene modifying moiety, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some embodiments, they are present in a bilayer structure, as micelles, or with a “collapsed” structure. Alternately, they are simply be interspersed in a solution, possibly forming aggregates that are not uniform in size or shape. Lipids are fatty substances which are, In some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids. In some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA.

In some embodiments, the system or any component of the system or any combination of the component of the system described herein can be delivered into the cell via biological methods such as the use of DNA and RNA vectors. Viral vectors, and especially retroviral vectors, have become the most widely used method for inserting genes into mammalian, e.g., human cells. Other viral vectors, In some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV comprises a serotype, including AAV1, AAV2, AAV4, AAV5, AAV6, AAV7, AAV8, AAV9, AAV10, AAV11, AAV12, or a combination thereof. Based on these initial serotypes. AAV capsid of each serotype can be engineered to make them better suited for biological functions, tissue or cell selection.

In some aspects, the system or any component of the system or any combination of the component of the system described herein comprises various gene editing methods to be used in the present disclosure to modify the cell described herein s, including CRISPR, RNA interference technology. TALENs (transcription activator-like (TAL) effector nucleases) and Zinc finger nucleases (ZFNs).

In some cases, CRISPR/Cas9 system is used to edit the genes of the immune cells. For example, CRISPR/Cas9 system can be used to knockout endogenous TCRs or cell surface markers (e.g., CS1, CD7, CD137) of the immune cells to generate the cell described herein s for T cell therapy. The CRISPR/Cas9 (clustered regular interspaced short palindromic repeats)/Cas (CRISPR-associated) system is a natural immune system unique to prokaryotes that is resistant to viruses or exogenous plasmids. The Type II CRISPR/Cas system has been applied in many eukaryotic and prokaryotic organisms as a direct genome-directed genome editing tool. The development of the CRISPR/Cas9 system has revolutionized the ability of people to edit DNA sequences and regulate the expression levels of target genes, providing a powerful tool for accurate genome editing of organisms. The simplified CRISPR/Cas9 system can comprise Cas9 protein and gRNA. The principle of action is that gRNA forms a Cas9-gRNA complex with Cas9 protein through its own Cas9 handle, and the base complementary pairing sequence of gRNA in the Cas9-gRNA complex is paired with the target sequence of the target gene by the principle of base complementary pairing. Cas9 uses its own endonuclease activity to cleave the target DNA sequence. Compared to traditional genome editing techniques, the CRISPR/Cas9 system has several distinct advantages: ease of use, simplicity, low cost, programmability, and the ability to edit multiple genes simultaneously. In some embodiments, the gene modifying moiety comprises a gRNA complexed with Cas to form a Cas/RNP. In some aspects, the gene modifying moiety is Cas9/RNP.

Proteins that bind to and are guided by a guide RNA to direct sequence specific cleavage or that, otherwise, bind nucleic acid sequences in a sequence specific way to trigger non-specific cleavage are consistent with the present disclosure. Such proteins include programmable endonucleases, such as programmable Cas endonucleases. Examples of programmable Cas endonucleases consistent with the present disclosure include Cas12a (or Cpf1), Cas12a, Cas12b, Cas12c, Cas12d, Cas12c, Cas12f, Cas12g, Cas12h, Cas12i, Cas13a, Cas13b, Cas14, Cas9, or others. In some cases, a site-specific endonuclease may include Cas12a (Cpf1), including any derivative thereof; any variant thereof; and any fragment thereof. Cas12a is classified as a class II, Type V CRISPR/Cas effector protein having about 1,300 amino acids. Cas12a is smaller than Cas9, Cas12a comprises two major domains such as REC and RuvC domains. Cas12a lacks the HNH endonuclease domain as in Cas9. Cas12a cleaves a double stranded DNA (dsDNA) immediately downstream from T-rich (5′-TTTN-3′) PAM. Cas12a generates a 4-5 nt-long 5′-overhang 20 nucleotides away from T-rich PAM. In some cases, the sticky ends produced by Cas12a enhance the efficiency of DNA replacement during HR. In some cases, a site-specific endonuclease may include Cas13a (C2c2). Alternatively, in some cases, a site-specific endonuclease may include Cas13b. Cas13 is an RNA-targeting endonuclease that exhibits a collateral effect of promiscuous RNAs activity upon target recognition.

In some embodiments, the method comprises contacting the cell with the system or any component of the system or any combination of the component of the system described herein described herein, where the cell comprises an immune cell or a stem cell. In some aspects, the cell is an immune cell described herein (e.g., a lymphocyte, a B cell, or a T cell) In some cases, the T cell can be cytotoxic T cell, alpha beta T cell, a gamma delta T cell, natural killer T cell, regulatory T cell, or T helper cell. In some aspects, the immune cell comprises an ILC. In some aspects, the stem cell is a hematopoietic stem cell or an iPSC. In some aspects, the iPSC can be derived into an immune cell or a T cell.

The cell described herein can be isolated from a sample from a donor who is not the subject in need of a treatment for a disease or condition described herein. The sample can be a bodily fluid or a tissue, including but not limited to, peripheral blood mononuclear cells, bone marrow, lymph node tissue, cord blood, thymus tissue, tissue from a site of infection, ascites, pleural effusion, spleen tissue, and tumors. In some cases, a sample comprises NK cells, NKT cells. T-cells or T-cell progenitor cells. For example, in some cases, the sample is an umbilical cord blood sample, a peripheral blood sample (e.g., a mononuclear cell fraction) or a sample from the subject comprising pluripotent cells. In some embodiments, a sample from the subject can be cultured to generate induced pluripotent stem (iPS) cells and these cells used to produce NK cells, NKT cells or T-cells. Cell samples may be cultured directly from the subject or may be cryopreserved prior to use. In some embodiments, obtaining a cell sample comprises collecting a cell sample. In other aspects, the sample is obtained by a third party. In still further aspects, a sample from a subject can be treated to purify or enrich the T-cells or T-cell progenitors in the sample. For example, the sample can be subjected to gradient purification, cell culture selection and/or cell sorting (e.g., via fluorescence-activated cell sorting (FACS)). The cell can be an NK cell. The NK cells can be obtained from peripheral blood, cord-blood, or other sources described herein. The NK cells can be derived from induced pluripotent stem cells. In some embodiments, a cell that can be utilized in a method provided herein can be positive or negative for a given factor. In some aspects, the cell can be prepared from a cell line.

In some embodiments, a cell provided herein can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell. CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated cell thereof. The examples of factors expressed by cells is not intended to be limiting, and a person having skill in the art will appreciate that a cell may be positive or negative for any factor known in the art. In some embodiments, a cell may be positive for two or more factors. For example, a cell may be CD4+ and CD8+.

In some embodiments, a cell may be negative for two or more factors. For example, a cell may be CD25−, CD44−, and CD69−. In some embodiments, a cell may be positive for one or more factors, and negative for one or more factors. For example, a cell may be CD4+ and CD8−. In some embodiments, a cellular marker provided herein can be utilized to select, enrich, or deplete a population of cells. In some embodiments, enriching comprises selecting a monocyte fraction. In some embodiments, enriching comprises sorting a population of immune cells from a monocyte fraction. In some embodiments, the cells may be selected for having or not having one or more given factors (e.g., cells may be separated based on the presence or absence of one or more factors). In some embodiments, the selected cells can also be transduced and/or expanded in vitro. The selected cells can be expanded in vitro prior to infusion. In some embodiments, selected cells can be transduced with a vector provided herein. It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells. In some cases, a cell can be a stem memory TSCM cell comprised of CD45RO (−), CCR7 (+), CD45RA (+), CD62L+ (L-selectin), CD27+, CD28+ and IL-7Ra+, stem memory cells can also express CD95, IL-2R13, CXCR3, and LFA-1, and show numerous functional attributes distinctive of memory cells. Cells provided herein can also be central memory TCM cells comprising L-selectin and CCR7, where the central memory cells can secrete, for example, IL-2, but not IFNy or IL-4. Cells can also be effector memory TEM cells comprising L-selectin or CCR7 and produce, for example, effector cytokines such as IFNy and IL-4.

In some aspects, the cell described herein can be an immune cell or stem cell. In some embodiments, the immune cell is a lymphocyte such as a T cell, a B cell, a natural killer (NK) cell, or a macrophage. In some aspects, the T cell is a cytotoxic T cell, a natural killer T cell, a regulatory T cell, or a T helper cell. In some embodiments, the cell to be modified is an immune cell comprising an innate lymphocyte (ILC). In some aspects, the cell modified by the method described herein is an immune cell derived from induced pluripotent stem cell (iPSC). In some embodiments, the immune cell is an iPSC derived T cell. In some embodiments, the immune cell is an iPSC derived natural killer T cell. In some embodiments, the cell modified by the method described herein is a stem cell. In some aspects, the stem cell can be a hematopoietic stem cell (HSC) or an induced pluripotent stem cell (iPSC).

In some embodiments, the cell described herein comprises a cell surface marker. The cell surface marker can be an immune cell antigen. The gene encoding the immune cell antigen of the immune cell used for preparing the cell described herein can be inactivated. Examples of immune cell antigens include, but are not limited to, CD2, CD3, CD4, CD5, CD7, CD8, CD 16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2F, CD279, CD314 (NKG2D), CD305, CD335 (NKP46), CD337, CD319 (CS1), TCRa, TCRf3 and SLAMF7. For example, in some cases, the gene encoding CD7 of the immune cell is inactivated.

In some embodiments, the method utilizing the system or any component of the system or any combination of the component of the system described herein increases a knockin efficiency of the chimeric polynucleotide described herein in a population of cells by at least 10%, at least 20%, at least 30%, at least 40%, or more compared to a nuclease (e.g., Cas) protein mediated knockin efficiency of a comparable chimeric polynucleotide in absence of chimeric being circular, having the at least one covalently closed end, or having the at least one gene modifying moiety targeting fragment in a comparable population of cells.

In some embodiments, the method utilizing the system or any component of the system or any combination of the component of the system described herein increases a knockin efficiency of the chimeric polynucleotide described herein in a population of cells by at least 10%, at least 20%, at least 30%, at least 40%, or more compared to a Cas protein mediated knockin efficiency of the same chimeric polynucleotide in absence of the at least one gene modifying moiety targeting fragment in a comparable population of cells.

In some embodiments, the method utilizing the system or any component of the system or any combination of the component of the system described herein increases an expression of the chimeric polynucleotide of the chimeric polynucleotide described herein in a population of cells by at least 10%, at least 20%, at least 30%, at least 40%, or more compared to a nuclease (e.g., Cas) protein mediated expression of the chimeric polynucleotide of a comparable chimeric polynucleotide in absence of chimeric being circular, having the at least one covalently closed end, or having the at least one gene modifying moiety targeting fragment in a comparable population of cells.

In some embodiments, the method utilizing the system or any component of the system or any combination of the component of the system described herein increases an expression of the chimeric polynucleotide of the chimeric polynucleotide described herein in a population of cells by at least 10%, at least 20%, at least 30%, at least 40%, or more compared to a Cas protein mediated expression of the chimeric polynucleotide of the same chimeric polynucleotide in absence of the at least one gene modifying moiety targeting fragment in a comparable population of cells.

In some embodiments, the method utilizing the system or any component of the system or any combination of the component of the system described herein increases a survival rate of the chimeric polynucleotide described herein in a population of cells by at least 10%, at least 20%, at least 30%, at least 40%, or more compared to a nuclease (e.g., Cas) protein mediated survival rate of a comparable chimeric polynucleotide in absence of chimeric being circular, having the at least one covalently closed end, or having the at least one gene modifying moiety targeting fragment in a comparable population of cells.

In some embodiments, the method utilizing the system or any component of the system or any combination of the component of the system described herein increases a survival rate of the chimeric polynucleotide described herein in a population of cells by at least 10%, at least 20%, at least 30%, at least 40%, or more compared to a Cas protein mediated survival rate of the same chimeric polynucleotide in absence of the at least one gene modifying moiety targeting fragment in a comparable population of cells.

In some cases, the cell described herein can exhibit (i) enhanced degree of persistence by remaining viable in vitro while in presence of cells that are heterologous to the cell described herein, including but not limited to heterologous T cells, heterologous NK cells and the mixture of the heterologous T cells and heterologous NK cells. (ii) enhanced degree of expansion, or (iii) enhanced cytotoxicity against a target cell comprising the antigen, compared to an additional engineered immune cell comprising the one or more CARs without the inactivation of the TCR, MHC molecule and/or immune cell antigen. In some cases, the cell described herein can be characterized by exhibiting two or more of (i) enhanced degree of persistence by remaining viable in vitro while in presence of cells that are heterologous to the cell described herein, including but not limited to heterologous T cells, heterologous NK cells and the mixture of the heterologous T cells and heterologous NK cells, (ii) enhanced degree of expansion, and (iii) enhanced cytotoxicity.

In some embodiments, the cell described herein can also comprise an enhancer moiety capable of enhancing one or more activities of the cell described herein. The enhancer moiety can be configured to constitutively upregulate one or more intracellular signaling pathways of the cell described herein. The one or more intracellular signaling pathways can be one or more cytokine signaling pathways. The enhancer moiety can be self-activating through self-oligomerizing. The enhancer moiety can be self-activating through self-dimerizing. The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSFIR, CTAL-4, TIM-3, CCL21, CCL19, TGFR beta, receptors for the same, functional fragments thereof, functional variants thereof, and combinations thereof.

In some aspects, the cell described herein can further comprise an inducible cell death moiety, which can effect suicide of the cell described herein upon contact with a cell death activator. The inducible cell death moiety can be selected from the group consisting of rapaCasp9, iCasp9, HSV-TK, ACD20, mTMPK, ACD19, RQR8, and EGFRt. In some cases, the inducible cell death moiety is EGFRt, and the cell death activator is an antibody or an antigen binding fragment thereof that binds EGFRt. In some cases, the inducible cell death moiety is HSV-TK, and the cell death activator is GCV. In some cases, the inducible cell death moiety is iCasp9, and the cell death activator is AP1903. The cell death activator can comprise a nucleic acid, a polynucleotide, an amino acid, a polypeptide, lipid, a carbohydrate, a small molecule, an enzyme, a ribosome, a proteasome, a variant thereof, or any combination thereof.

In some aspects, the cell described herein provided herein can comprise a chimeric polypeptide comprising (i) an enhancer moiety capable of enhancing one or more activities of the cell described herein, and (ii) an inducible cell death moiety capable of effecting death of the cell described herein upon contacting the chimeric polypeptide with a cell death activator, wherein the enhancer moiety is linked to the inducible cell death moiety. In some cases, the enhancer moiety and the inducible moiety may be linked by a linker. The linker can be a cleavable linker, for example, a self-cleaving peptide.

In some embodiments, the cell described herein can further comprise at least one heterologous polypeptide comprising at least one heterologous receptor. In some aspects, the heterologous receptor is a chimeric polypeptide receptor (CPR) comprising a binding moiety, wherein the binding moiety comprises (i) a first antigen binding domain, which first antigen binding domain suppresses or decreases a subject's immune response toward the cell described herein when administered into the subject and (ii) a second antigen binding domain capable of binding to a disease-associated antigen. An individual CPR of the one or more CPRs can comprise (i) the first antigen binding domain. (ii) the second antigen binding domain, or (iii) both the first antigen binding domain and the second antigen binding domain. A CPR of the one or more CPRs can further comprise a transmembrane domain and an intracellular signaling region. In some cases, the one or more CPRs in the cell described herein are one or more chimeric antigen receptors (CARs) or engineered T cell receptors (TCRs). In some cases, the cell described herein s comprise both CARs and engineered TCRs. In some embodiments, the at least one heterologous receptor comprises at least one chimeric antigen receptor (CAR), where each CAR of the at least one CAR comprise a hinge, a transmembrane domain, a costimulatory, and an intracellular signaling region.

The engineered TCR can be a TCR fusion protein. For example, the TCR fusion protein can comprise a heterologous antigen binding domain fused to one or more subunits of a TCR complex. In some cases, the TCR fusion protein can comprise a TCR subunit comprising at least a portion of a TCR extracellular domain and a TCR intracellular domain; and an antibody domain comprising an antigen binding domain, where the TCR subunit and the antibody domain are linked. The TCR fusion protein can incorporate into a TCR complex when expressed in a T cell. In some cases, the TCR fusion protein can further comprise a TCR transmembrane domain. The TCR extracellular domain, the TCR intracellular domain, or the TCR transmembrane domain can be derived from TCR alpha chain, TCR beta chain, TCR gamma chain, TCR delta chain, CD3 epsilon, CD3 gamma, CD3 delta or CD3 zeta. In some cases, an endogenous TCR of the cell described herein comprising an engineered TCR is inactivated.

In some cases, the cell described herein comprising inactivated endogenous TCR may not cause GVHD. For example, a gene encoding an endogenous TCR subunit can be inactivated. For another example, a gene encoding an endogenous TCR subunit may be mutated such that an endogenous TCR may not be formed.

CARs can comprise an extracellular antigen recognition region, for example, a scFv (single-chain variable fragment), a transmembrane region, and an intracellular costimulatory signal region. The extracellular domain of CARs can recognize a specific antigen and then transduce the signal through the intracellular domain, causing T cell activation and proliferation, cytolysis toxicity, and secretion of cytokines, thereby eliminating target cells. The patient's autologous T cells (or heterologous donors) can be first isolated, activated and genetically engineered to produce CAR-T cells, which can be then injected into the same patient. In this way, the probability of graft-versus-host disease may be decreased, and the antigen can be recognized by T cells in a non-MHC-restricted manner. In addition, a CAR-T can treat all cancers that express the antigen.

In some aspects, the cell described herein can target both disease-associated antigen (e.g., tumor-associated antigen or tumor cell marker) and immune cell antigen (e.g., CD3, CD7 or CD137) through bispecific or multivalent CAR(s). For example, the present disclosure provides an engineered immune cell that can target a tumor cell marker and an immune cell antigen such as CD3. The endogenous TCR can be inactivated (e.g., disrupted, inhibited, knocked out or silenced). The CAR-T of the present disclosure which targets the tumor cell marker and the immune cell antigen can eliminate positive tumor cells and clear host immune cell antigen positive T and NK cells, thereby avoiding host rejection (HVG). In the present disclosure, the endogenous TCR of the cell described herein can be knocked out, and graft-versus-host disease (GVHD) can be prevented, thereby preparing a general-purpose or universal CAR-T (UCAR-T) cell. The cell described herein can be derived from an autologous T cell or an allogeneic T cell. Moreover, the cell described herein can comprise a cell suicide element (e.g., inducible cell death moiety), and the CAR-T can be inactivated/cleared at any time to decrease side effects. In some cases, the cell described herein can further comprise an enhancer moiety. The enhancer moiety can regulate one or more activities of the cell described herein when the cell described herein is administered to a subject. For example, the enhancer moiety can be a cytokine (e.g., IL-5 or IL-7) or a cytokine receptor (e.g., IL-5R or IL-7R). The enhancer moiety can enhance a signaling pathway within the cell described herein, for example, STAT5 signaling pathway. In some embodiments, the cell described herein comprises a bispecific CAR targeting both CD19 and CD3. The cell described herein show in this example can further comprise an inducible cell death moiety such as a truncated epidermal growth factor receptor (EGFRt or tEGFR, which can be used interchangeably herein. The inducible cell death moiety or the enhancer moiety can be introduced in the immune cell via a separate expression vector. In some cases, the inducible cell death moiety and the enhancer moiety may be introduced into the immune cell via an expression vector comprising sequences encoding both moieties. In some cases, the inducible cell death moiety and the enhancer moiety are linked and are expressed as a chimeric polypeptide. The application of the cell described herein can be used for cell-based therapy for treating a disease or condition (e.g., cancer) of a subject, be prepared in large-scale in advance to avoid GVHD and HvG, decrease treatment costs, inactivate CAR-T at any time if necessary, decrease side effects of immunotherapy, and ensure product safety.

### Methods of Treatment

Disclosed herein, In some embodiments, are methods of using the system described herein, the chimeric polynucleotide described herein, or the cell modified described herein. In some embodiments, the methods include treating a disease or condition of a subject by administering the system described herein, the chimeric polynucleotide described herein, the cell modified described herein, or a pharmaceutical composition described herein to the subject. In some embodiments, administration is by any suitable mode of administration, including systemic administration (e.g., intravenous, inhalation, etc.). In some embodiments, the subject is human.

In some embodiments, the system described herein, the chimeric polynucleotide described herein, the cell modified described herein, or the pharmaceutical composition described herein is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time.

In some embodiments, the system described herein, the chimeric polynucleotide described herein, the cell modified described herein, or the pharmaceutical composition described herein is administered in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration. e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the system described herein, the chimeric polynucleotide described herein, the cell modified described herein, or the pharmaceutical composition described herein is administered by intravenous infusion. In some embodiments, the composition is administered by slow continuous infusion over a long period, such as more than 24 hours. In some embodiments, the composition is administered as an intravenous injection or a short infusion.

The system described herein, the chimeric polynucleotide described herein, the cell modified described herein, or the pharmaceutical composition described herein may be administered in a local manner, for example, via injection of the agent directly into an organ, optionally in a depot or sustained release formulation or implant. A composition may be provided in the form of a rapid release formulation, in the form of an extended release formulation, or in the form of an intermediate release formulation. A rapid release form may provide an immediate release. An extended release formulation may provide a controlled release or a sustained delayed release. In some embodiments, a pump may be used for delivery of the composition. In some embodiments, a pen delivery device may be used, for example, for subcutaneous delivery of a composition of the disclosure.

The system described herein, the chimeric polynucleotide described herein, the cell modified described herein, or the pharmaceutical composition described herein can be administered in conjunction with other therapies, for example, an antiviral therapy, a chemotherapy, an antibiotic, a cell therapy, a cytokine therapy, or an anti-inflammatory agent. In some embodiments, a circular polyribonucleotide or the antibody or the antigen-binding fragment thereof described herein may be used singly or in combination with one or more therapeutic agents as a component of mixtures. In some embodiments, a linear polyribonucleotide or the antibody or the antigen-binding fragment thereof described herein may be used singly or in combination with one or more therapeutic agents as a component of mixtures.

The system described herein, the chimeric polynucleotide described herein, the cell modified described herein, or the pharmaceutical composition described herein can be administered before, during, or after the occurrence of a disease or condition, and the timing of administering the composition containing a therapeutic agent may vary. In some cases, the cell or the pharmaceutical composition may be used as a prophylactic and may be administered continuously to subjects (e.g., the subject for immunization or the subject for treatment) with a susceptibility to an infection by a pathogen or a propensity to a condition or disease associated with the pathogen. Prophylactic administration may lessen a likelihood of the occurrence of the infection, disease, or condition, or may decrease the severity of the infection, disease, or condition.

The system described herein, the chimeric polynucleotide described herein, the cell modified described herein, or the pharmaceutical composition described herein can be administered to a subject before the onset of the symptoms. The composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent may be administered after (e.g., as soon as is practicable after) the onset of a disease or condition is detected or suspected. A therapeutic agent may be administered after (e.g., as soon as is practicable after) a potential exposure to a coronavirus, for example, after a subject (e.g., the subject for immunization or the subject for treatment) has contact with an infected subject, or learns they had contact with an infected subject that may be contagious.

Actual dosage levels of an agent of the disclosure (e.g., the system described herein, the chimeric polynucleotide described herein, the cell modified described herein, or the pharmaceutical composition described herein) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally decreased or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The system described herein, the chimeric polynucleotide described herein, the cell modified described herein, or the pharmaceutical composition described herein can be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

In some embodiments, a dose may be based on the number of the cells per kilogram of body weight of a subject. In some embodiments, a dose may be is administered in a dosage amount of between about 1000 cells/kg body weight to about 1000000000000 cells/kg body weight, about 1,000 cells/kg body weight to about 1,000,000,000,000 cells/kg body weight. In some embodiments, a dose may be is administered in a dosage amount of between about 1000 cells/kg body weight to about 1000000000000 cells/kg body weight, about 1,000 cells/kg body weight to about 10,000 cells/kg body weight, about 1,000 cells/kg body weight to about 100,000 cells/kg body weight, about 1,000 cells/kg body weight to about 1,000,000 cells/kg body weight, about 1,000 cells/kg body weight to about 10,000,000 cells/kg body weight, about 1,000 cells/kg body weight to about 100,000,000 cells/kg body weight, about 1,000 cells/kg body weight to about 1,000,000,000 cells/kg body weight, about 1,000 cells/kg body weight to about 10,000,000,000 cells/kg body weight, about 1,000 cells/kg body weight to about 100,000,000,000 cells/kg body weight, about 1,000 cells/kg body weight to about 1,000,000,000,000 cells/kg body weight, about 10,000 cells/kg body weight to about 100,000 cells/kg body weight, about 10,000 cells/kg body weight to about 1,000,000 cells/kg body weight, about 10,000 cells/kg body weight to about 10,000,000 cells/kg body weight, about 10,000 cells/kg body weight to about 100,000,000 cells/kg body weight, about 10,000 cells/kg body weight to about 1,000,000,000 cells/kg body weight, about 10,000 cells/kg body weight to about 10,000,000,000 cells/kg body weight, about 10,000 cells/kg body weight to about 100,000,000,000 cells/kg body weight, about 10,000 cells/kg body weight to about 1,000,000,000,000 cells/kg body weight, about 100,000 cells/kg body weight to about 1,000,000 cells/kg body weight, about 100,000 cells/kg body weight to about 10,000,000 cells/kg body weight, about 100,000 cells/kg body weight to about 100,000,000 cells/kg body weight, about 100,000 cells/kg body weight to about 1,000,000,000 cells/kg body weight, about 100,000 cells/kg body weight to about 10,000,000,000 cells/kg body weight, about 100,000 cells/kg body weight to about 100,000,000,000 cells/kg body weight, about 100,000 cells/kg body weight to about 1,000,000,000,000 cells/kg body weight, about 1,000,000 cells/kg body weight to about 10,000,000 cells/kg body weight, about 1,000,000 cells/kg body weight to about 100,000,000 cells/kg body weight, about 1,000,000 cells/kg body weight to about 1,000,000,000 cells/kg body weight, about 1,000,000 cells/kg body weight to about 10,000,000,000 cells/kg body weight, about 1,000,000 cells/kg body weight to about 100,000,000,000 cells/kg body weight, about 1,000,000 cells/kg body weight to about 1,000,000,000,000 cells/kg body weight, about 10,000,000 cells/kg body weight to about 100,000,000 cells/kg body weight, about 10,000,000 cells/kg body weight to about 1,000,000,000 cells/kg body weight, about 10,000,000 cells/kg body weight to about 10,000,000,000 cells/kg body weight, about 10,000,000 cells/kg body weight to about 100,000,000,000 cells/kg body weight, about 10,000,000 cells/kg body weight to about 1,000,000,000,000 cells/kg body weight, about 100,000,000 cells/kg body weight to about 1,000,000,000 cells/kg body weight, about 100,000,000 cells/kg body weight to about 10,000,000,000 cells/kg body weight, about 100,000,000 cells/kg body weight to about 100,000,000,000 cells/kg body weight, about 100,000,000 cells/kg body weight to about 1,000,000,000,000 cells/kg body weight, about 1,000,000,000 cells/kg body weight to about 10,000,000,000 cells/kg body weight, about 1,000,000,000 cells/kg body weight to about 100,000,000,000 cells/kg body weight, about 1,000,000,000 cells/kg body weight to about 1,000,000,000,000 cells/kg body weight, about 10,000,000,000 cells/kg body weight to about 100,000,000,000 cells/kg body weight, about 10,000,000,000 cells/kg body weight to about 1,000,000,000,000 cells/kg body weight, or about 100,000,000,000 cells/kg body weight to about 1,000,000,000,000 cells/kg body weight. In some embodiments, a dose may be is administered in a dosage amount of between about 1000 cells/kg body weight to about 1000000000000 cells/kg body weight, about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, a dose may be is administered in a dosage amount of between about 1000 cells/kg body weight to about 1000000000000 cells/kg body weight, at least about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, or about 100,000,000,000 cells/kg body weight. In some embodiments, a dose may be is administered in a dosage amount of between about 1000 cells/kg body weight to about 1000000000000 cells/kg body weight, at most about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years.

In some embodiments, the method described herein treats a disease or condition in a subject, there the method comprises administering the cell or the pharmaceutical composition descried herein without triggering innate immune response or GVHD in the subject being treated. In somebodies, the cell or the pharmaceutical composition treats a disease or condition in a subject in need thereof, where the disease or condition is cancer. Non-limiting example of cancer includes Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer. Birt Hogg Dube Syndrome, Bladder Cancer, Bone Cancer, Brain Tumor, Breast Cancer. Bronchial Tumors, Burkitt Lymphoma. Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer. Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer. Histiocytosis. Langerhans Cell. Hodgkin's Lymphoma. Hypopharyngeal Cancer. Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer. Liver Cancer. Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms. Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma. Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sezary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer. Vascular Tumors, Vulvar Cancer, Wilms Tumor, or a combination thereof.

In some embodiments, the cell described herein (e.g., the modified cell expressing at least one transgene described herein) can target a cancer cell or a tumor cell for treating the disease or condition in the subject. Non-limiting examples of cancer cell or tumor cell, as used in the present disclosure, may include cell of cancer including Acanthoma, Acinic cell carcinoma, Acoustic neuroma, Acral lentiginous melanoma, Acrospiroma, Acute eosinophilic leukemia. Acute lymphoblastic leukemia. Acute megakaryoblastic leukemia. Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma. Adenoid cystic carcinoma. Adenoma. Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma. Anaplastic thyroid cancer. Angioimmunoblastic T-cell lymphoma. Angiomyolipoma. Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma. Biliary tract cancer, Bladder cancer. Blastoma. Bone Cancer. Bone tumor. Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma. Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma. Cutaneous T-cell lymphoma, Degos disease, Dermatofibrosarcoma protuberans, Dermoid cyst. Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone. Glioblastoma multiforme, Glioma, Gliomatosis cerebri, Glomus tumor. Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma. Hepatosplenic T-cell lymphoma. Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma. Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia. Leukemia, Lip and Oral Cavity Cancer, Liposarcoma, Lung cancer, Lutcoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma. Malignant peripheral nerve sheath tumor. Malignant rhabdoid tumor. Malignant triton tumor, MALT lymphoma. Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma. Mixed Mullerian tumor, Monocytic leukemia, Mouth Cancer, Mucinous tumor. Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer. Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer. Osteosarcoma, Osteosarcoma, Ovarian Cancer, Ovarian cancer. Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma. Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer. Pseudomyxoma peritonei. Rectal Cancer. Renal cell carcinoma. Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Solid tumor, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor. Spinal tumor, Splenic marginal zone lymphoma, Squamous cell carcinoma. Stomach cancer, Superficial spreading melanoma, Supratentorial Primitive Neuroectodermal Tumor. Surface epithelial-stromal tumor. Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm. Uterine sarcoma, Uveal melanoma, Vaginal Cancer, Verner Morrison syndrome, Verrucous carcinoma. Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoictic lineage, such as a lymphoma.

### Pharmaceutical Compositions

Described herein is a pharmaceutical composition comprising a therapeutic agent (e.g., the system described herein, the chimeric polynucleotide described herein, or the cell modified described herein). In some aspects, the pharmaceutical composition comprises a pharmaceutically acceptable: carrier, excipient, or diluent. In some aspects, the pharmaceutical composition described herein includes at least one additional active agent other than the cell described herein. In some aspects, the at least one additional active agent is a chemotherapeutic agent, cytotoxic agent, cytokine, growth-inhibitory agent, anti-hormonal agent, anti-angiogenic agent, or checkpoint inhibitor.

In practicing the methods of treatment or use provided herein, therapeutically effective amount of pharmaceutical composition described herein is administered to a mammal having a disease, disorder, or condition to be treated, e.g., cancer. In some aspects, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition including a therapeutic agent may be manufactured in a conventional manner, such as, by way of example only, by means of conventional mixing, dissolving, granulating, dragee-making, levigating, emulsifying, encapsulating, entrapping or compression processes.

The pharmaceutical composition may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some aspects, therapeutic agents exist in unsolvated form or in solvated forms with pharmaceutically acceptable solvents such as water, ethanol, and the like. The solvated forms of the therapeutic agents are also considered to be disclosed herein.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some aspects, pharmaceutical composition described herein benefits from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid. (f) 0.003% to about 0.02% w/v polysorbate 80. (g) 0.001% to about 0.05% w/v, polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical composition described herein can be formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some aspects, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a pharmaceutical composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections may involve bolus injection or continuous infusion. Pharmaceutical composition for injection may be presented in unit dosage form, e.g., in ampoules or in multi dose containers, with an added preservative. The pharmaceutical composition described herein may be in a form suitable for parenteral injection as a sterile suspensions, solutions, or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist, or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparation for oral use is obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some aspects, dyestuffs or pigments are added to the tablets or dragee coatings for identification or to characterize different combinations of active therapeutic agent doses.

In some aspects, the pharmaceutical composition of the exogenous therapeutic agents is in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some aspects, stabilizers are added. A capsule may be prepared, for example, by placing the bulk blend of the formulation of the therapeutic agent inside of a capsule. In some aspects, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to cating.

Pharmaceutical composition for oral administration can be in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some aspects, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some aspects, the film coating may provide a delayed release of a therapeutic agent from the formulation. In other embodiments, the film coating aids in patient compliance. Film coatings typically range from about 1% to about 3% of the tablet weight. In some aspects, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some aspects, the individual unit dosages include film coatings. These formulations are manufactured by conventional formulation techniques.

In another aspect, dosage forms include microencapsulated formulations. In some aspects, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some aspects, the aqueous dispersions further include a crystal-forming inhibitor.

In some aspects, the pharmaceutical composition described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some aspects, SEDDS provides improvements in the bioavailability of hydrophobic active ingredients.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some aspects, a composition described herein is in a form suitable for parenteral injection as a sterile suspensions, solutions, or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

In some aspects, the pharmaceutical composition can be provided that include particles of a therapeutic agent and at least one dispersing agent or suspending agent for oral administration to a subject. The formulations may be a powder and/or granule for suspension, and upon admixture with water, a substantially uniform suspension is obtained.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the pharmaceutical composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

### Kits

Described herein, in some aspects, are kits for using the system described herein, the chimeric polynucleotide described herein, or the cell modified described herein for practicing the method described herein. In some aspects, the kits disclosed herein may be used to treat a disease or condition in a subject. In some aspects, the kits comprise an assemblage of materials or components apart from the cell.

In some aspects, the kit comprises the components for assaying the number of units of a biomolecule (e.g., a therapeutic agent) synthesized, and/or released or expressed on the surface by the cell described herein. In some aspects, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some aspects, the kit is configured particularly for the purpose of treating mammalian subjects. In some aspects, the kit is configured particularly for the purpose of treating human subjects.

Instructions for use may be included in the kit. In some aspects, the kit comprises instructions for administering the cell to a subject in need thereof. In some aspects, the kit comprises instructions for further engineering the composition to express a biomolecule (e.g., a therapeutic agent). In some aspects, the kit comprises instructions thawing or otherwise restoring biological activity of the cell, which may have been preserved during storage or transportation. In some aspects, the kit comprises instructions for measure viability of the preserved cell, to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together. B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Cell Modification

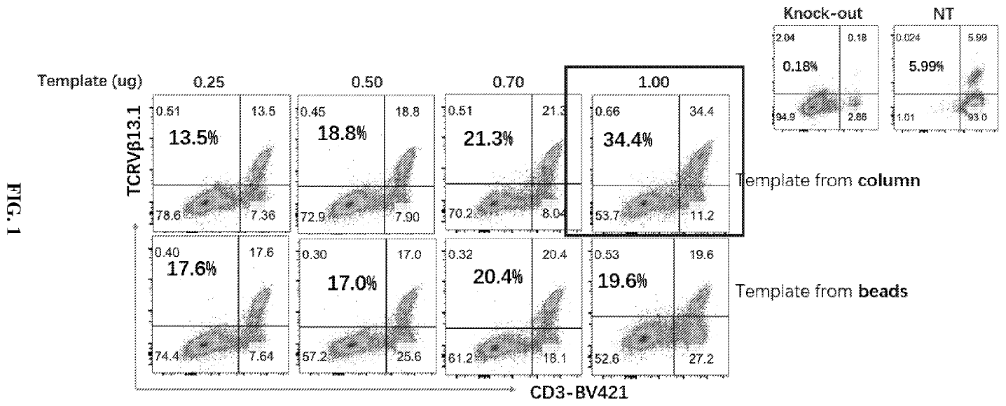

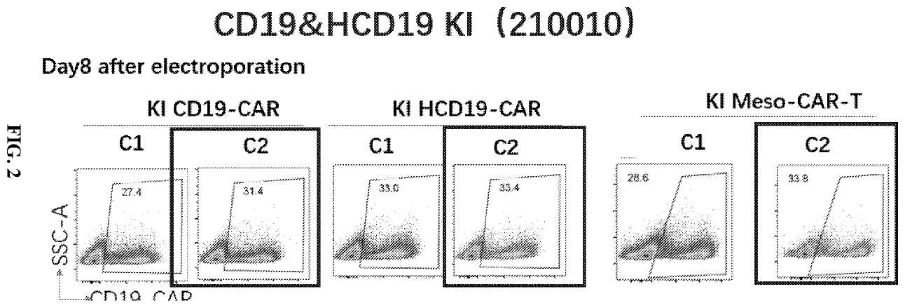

Cells described herein were contacted with the system or any component of the system or any combination of the component of the system described herein for knockin of the chimeric receptor or the MHC protein. The modified cells expressing the chimeric receptor or the MHC protein were examined and illustrated in FIG. 1-FIG. 14. For the knockin or knockout experiments, the cells to be modified were first activated on day 0. Knockin or knockout experiments were carried out on day 2. On day 10, flow cytometry was conducted to determine the efficiency of knockin or knockout experiment. Table 1 illustrates the ratio between RNP and various other components of the system described herein for modifying the cells. Table 2 illustrates exemplary amount of chimeric polynucleotide contacted with cells for generating the data in in FIG. 1-FIG. 14. Table 3 illustrates exemplary materials for practicing the methods described herein. Table 4 illustrates exemplary amount of chimeric polynucleotide contacted with ten million cells to be modified. Table 5 illustrates exemplary electroporation protocol of the Lonza 4D-Nucleofector protocol. Table 6 illustrates exemplary maxcyte program.

By using CRISPR/Cas9 for knocking in the chimeric polynucleotide described herein (via homology directed repair, HDR), the percentage of knockin (KI) single CAR gene was about 30% to 50% based on the data of knockin at TRAC and B2M locus. Simultaneously knockin of two chimeric polynucleotides (e.g., at TRAC locus and at B2M locus) was effective more than 20%. The percentage of knockin of NY-ESO-1 specific TCR at TRAC locus was 34.4% via using chimeric polynucleotide comprising double stranded DNA with the at least one gene modifying moiety targeting fragment. Using chimeric polynucleotide comprising double stranded DNA with the at least one gene modifying moiety targeting fragment, the knockin efficiency of CD19 specific CAR gene at TRAC locus was 31.4%; the knockin efficiency of humanized CD19 specific CAR gene at TRAC locus was 33.4%; and the knockin efficiency of mesothelin specific CAR gene at TRAC locus was 33.8%. The knockin efficiency of GC020 at TRAC locus was 41.1%. The knockin efficiency of GC012 at TRAC locus was 22.5%. The knockin efficiency of HLA-E at B2M locus using chimeric polynucleotide comprising double stranded DNA with the at least one gene modifying moiety targeting fragment was 37.7%, and the efficiency was up to 54.9% while using chimeric polynucleotide comprising double stranded DNA with the at least one gene modifying moiety targeting fragment and telomeric ends. The knockin efficiency of GC012HL at TRAC locus chimeric polynucleotide comprising double stranded DNA with the at least one gene modifying moiety targeting fragment with telomeric ends template was 34.6%. The efficiency of simultaneously knockin of GC012HL at TRAC locus and HLA-E at B2M locus was 21.7% and 50.1% for the GC012HL knockin. The efficiency of simultaneously knockin of GC012HL at TRAC locus and HLA-E at B2M locus was 31.8%. The chimeric polynucleotide to be knocked in by the system and method described herein can be large DNA template (e.g., more than five thousand nucleotide base pairs).

TABLE 1
Non-limiting example of RNP ratio

| RNP | 1 | 2 | 3 | 4 | 5 | 6 | 7 | 8 |
|---|---|---|---|---|---|---|---|---|
| Cas9 (ul) | 0.33 | 0.33 | 0.33 | 0.165 | 0.33 | 0.33 | 0.33 | 0.17 |
| sgRNA (ul) | 0.8 | 0.8 | 0.8 | 0.4 | 0.5 | 0.5 | 0.5 | 0.25 |
| PGA (ul) | 0.8 | 0.6 | 0.4 | 0.4 | 0.83 | 0.44 | 0.32 | 0.42 |
| 20 ul/R | 1.93 | 1.73 | 1.53 | 0.965 | 1.66 | 1.27 | 1.15 | 0.83 |
| 100 ul/R(X5) | 9.65 | 8.65 | 7.65 | 4.825 | 8.3 | 6.35 | 5.75 | 4.15 |

TABLE 2
Non-limiting example of amount of chimeric
polynucleotide for modifying cells

| GC012HL (ug) | 0.5 | 0.7 | 1.0 | 1.3 | 1.5 | 2.0 | 2.5 | 3.0 |
|---|---|---|---|---|---|---|---|---|
| HLA-E (ug) | 0.5 | 0.7 | 1.0 | 1.3 | 1.5 | 2.0 | 2.5 | 3.0 |

TABLE 3
Exemplary materials for practicing the methods described herein

| Reagents | Vendor | Catalog number |
|---|---|---|
| X-VIVO 15 | LONZA | 04-418Q |
| IL-2 | Peprotech | AF-200-02-1MG |
| HSA | Baxter | 750306 |
| Serum replacement | Gibco | A3020902 |
| sgRNA-TRAC | Gene script | Easy edit |
| sgRNA-B2M | Gene script | Easy edit |
| Cas9-HiFi | IDT | 1081061 |
| Duplex buffer | IDT | 1072507 |
| PGA | Sigma | P4761 |
| P3 Primary | Lonza | V4XP-23024 |
| Cell 4D- | | |
| NucleofectorTM | | |
| X Kit | | |
| PGA | Sigma | P4761 |

TABLE 4
Exemplary amount of chimeric polynucleotide contacted
with ten million cells to be modified

| GC012HL(ug) | 0.2 | 0.5 | 0.7 | 1 | 1.3 | 1.5 | 2 | 2.5 | 3 |
|---|---|---|---|---|---|---|---|---|---|
| HLA-E(ug) | 0.2 | 0.5 | 0.7 | 1 | 1.3 | 1.5 | 2 | 2.5 | 3 |

TABLE 5
Exemplary electroporation protocol of
the Lonza 4D-Nucleofector protocol

| | | | | |
|---|---|---|---|---|
| Programs | EH-115 | DS-137 | EO-115 | FI-115 |
| | EC-138 | EC-156 | EF-138 | EO-151 |

TABLE 6
Exemplary Maxcyte program

| | | | |
|---|---|---|---|
| Programs | CSB-high | CSB T high | Expanded T |
| | energy 1 | energy 2-2 | cell 3 |

| | |
|---|---|
| Expanded T cell 4 | Expanded T cell 5 |

### Example 2. Knockin of NY-ESO-1 Targeting TCR at TRAC Locus

In order to detect the knockin efficiency of NY-ESO-1 specific TCR at TRAC locus, T and TRAC knock-out T cells were used as negative control. “Template from column” denotes chimeric polynucleotide obtained from column purification. “Template from beads” denotes chimeric polynucleotide obtained from purification from magnetic beads. One million cells (in a final volume of 20 μl) were contacted with 0.25 μg, 0.50 μg, 0.70 μg, or 1.00 μg of chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). Flow cytometry analysis showed that CD3 was knocked out in 94.9% of the cells; and 0.18% of the cells were positive for both TCRVβ13.1 and CD3.

Knockin efficiency was detected on specific day after electroporation by flow cytometry, as shown in FIG. 1, the CD3 knock out efficiency was 94.9%, while TCRVβ13.1 and CD3 double positive percentage was 0.18% of TRAC knockout control. The highest knockin efficiency of NY-ESO-1 specific TCR was 34.4% with the chimeric polynucleotide usage of 1 μg.

### Example 3. Knockin of CD19 Targeting CAR at TRAC Locus

The knockin was mediated by chimeric polynucleotide comprising the modifying moiety targeting fragment described herein at both 5′ end and 3′ end of the chimeric polynucleotide. The chimeric polynucleotide was not covalently closed at both 5′ end and 3′ end of the chimeric polynucleotide. The knockin yielded: a population of 31.4% cells expressing the CD19 targeting CAR (CD-19-CAR) at the TRAC locus; a population of 33.4% cells expressing the humanized CD19 targeting CAR (HCD19-CAR) at the TRAC locus and a population of 33.8% cells expressing the Mesothelin targeting CAR (Meso-CAR-T) at the TRAC locus. One million cells (in a final volume of 20 μl) were contacted with 0.20 μg or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

Knockin efficiency was detected on specific day after electroporation by flow cytometry. As shown in FIG. 2, the highest knockin efficiency of CD19 targeting CAR and humanized CD19 CAR and mesothelin CAR was 31.4%, 33.4%, and 33.8% respectively.

### Example 4. Knockin of GC020 or GC012 at TRAC

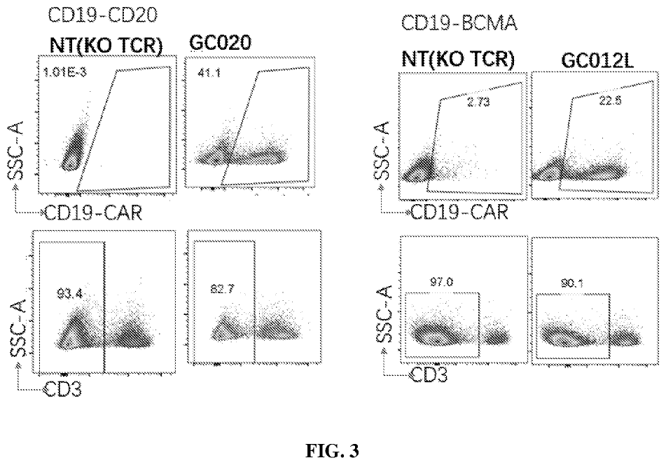

The knockin was mediated by chimeric polynucleotide comprising the modifying moiety targeting fragment described herein at both 5′ end and 3′ end of the chimeric polynucleotide. The chimeric polynucleotide was not covalently closed at both 5′ end and 3′ end of the chimeric polynucleotide. The knockin yielded: a population of 41.1% cells expressing the dual CAR of CD19 and CD20 via the knockin at the TRAC genomic locus (with 82.7% of the cells exhibiting TRAC knockout); and a population of 22.5% cells expressing the dual CAR of CD19 via the knockin at the TRAC genomic locus (with 90.1% of the cells exhibiting TRAC knockout). One million cells (in a final volume of 20 μl) were contacted with 0.20 μg or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). Knockin efficiency was detected on specific day after electroporation through flow cytometry. As shown in FIG. 3, the knockin efficiency of GC020 was 41.1% and 22.5% for GC012, while the knockout efficiency was 82.7% and 90.1% respectively.

### Example 5. Knockin of HLA-E

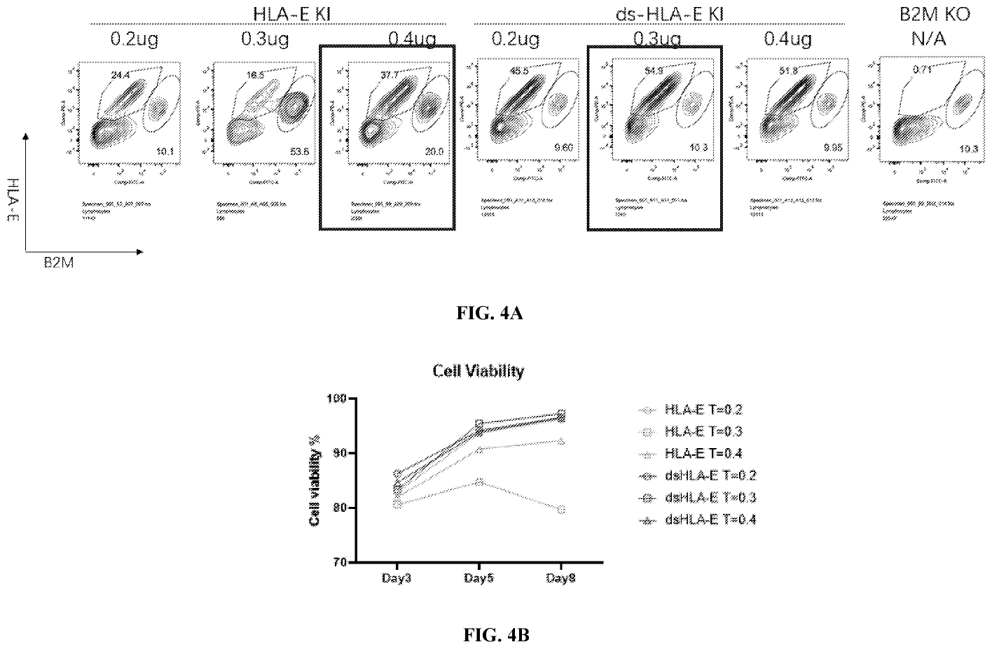

FIG. 4A illustrates knocking in of HLA-E into cells by the system described herein. The knockin was mediated by chimeric polynucleotide comprising the modifying moiety targeting fragment described herein at both 5′ end and 3′ end of the chimeric polynucleotide. The chimeric polynucleotide was further modified to comprise both covalently closed 5′ end and covalently closed 3′ end. The knockin mediated by chimeric polynucleotide comprising the modifying moiety targeting fragment described herein at both ends but without covalently closed 5′ end and 3′ end yielded a population of 37.7% cells expressing HLA-E (HLA-E KI). The knockin mediated by chimeric polynucleotide comprising the modifying moiety targeting fragment described herein at both ends and with covalently closed 5′ end and 3′ end yielded a population of 54.9% cells expressing HLA-E (ds-HLA-E KI). One million cells (in a final volume of 20 μl) were contacted with 0.20 μg, 0.30 μg, or 0.40 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). FIG. 4B illustrates cell viability measurements showing that the cells modified with chimeric polynucleotide comprising the modifying moiety targeting fragment at both ends and with covalently closed 5′ end and 3′ end were equally or more viable than cells modified with chimeric polynucleotide comprising the modifying moiety targeting fragment described herein at both ends but without covalently closed 5′ end and 3′ end across eight days. Knockin efficiency was detected on specific day after electroporation by flow cytometry. As shown in FIG. 4A, the highest knockin efficiency of HLA-E was 37.7% with the chimeric polynucleotide concentration at 0.4 μg, and the highest knockin efficiency of ds-HLA-E was up to 54.9% when the chimeric polynucleotide concentration was 0.3 μg. The viability of knockin ds-HLA-E was higher than HLA-E (FIG. 4B).

### Example 6. Knockin of GC012HL at TRAC Locus

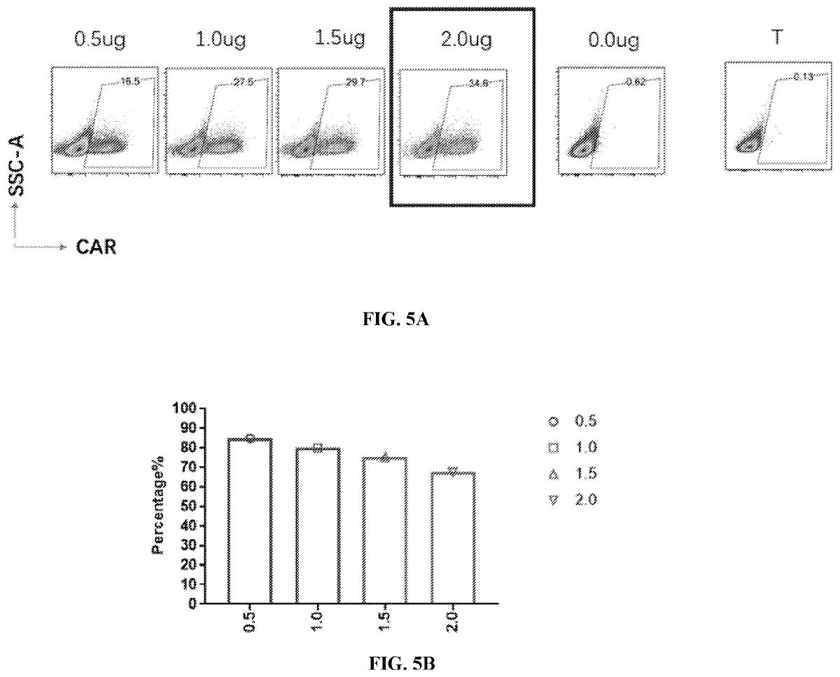

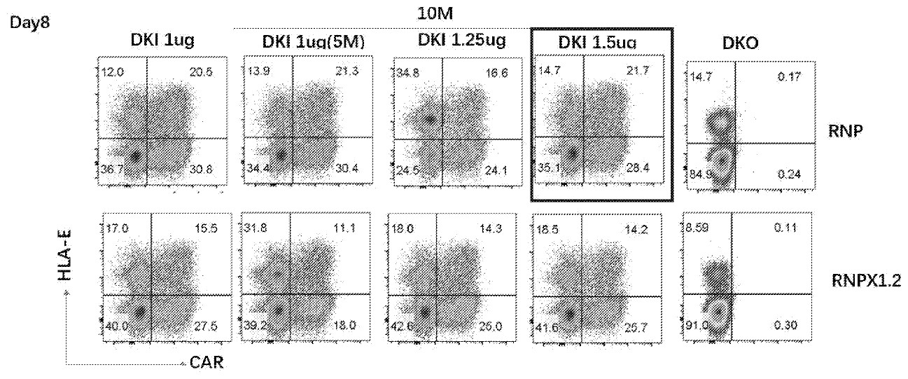

The knockin was mediated by chimeric polynucleotide (at an amount of 0.5 μg, 1.0 μg, 1.5 μg, or 2.0 μg, FIG. 5A) comprising the modifying moiety targeting fragment at both 5′ end and 3′ end and covalently closed 5′ end and covalently closed 3′ end, 34.6% of the cells expressed the dual CAR. One million cells (in a final volume of 20 μl) were contacted with 0.50 μg, 1.0 μg, 1.5 μg, or 2.0 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). FIG. 5B illustrates cell viability of the knocked in cells after three days.

### Example 7. Simultaneously Knockin of GC012HL at TRAC Locus and HLA-E at B2M Locus

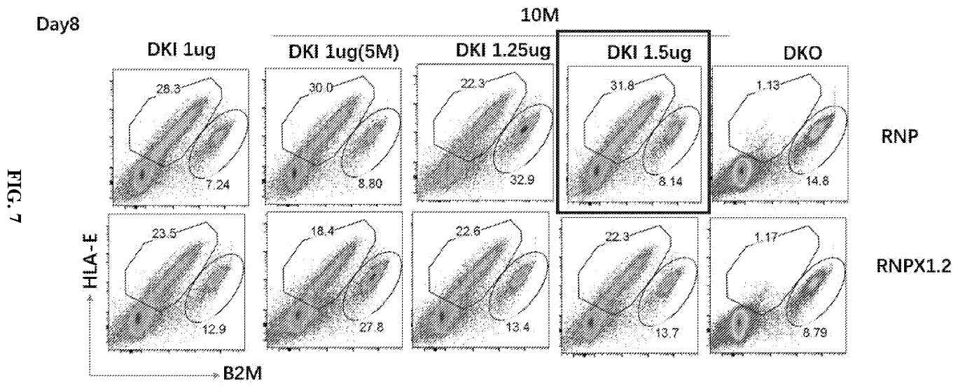

FIG. 6 illustrates knocking in of both dual CAR (Dual-CAR-T) and HLA-E at TRAC locus and at B2M locus respectively, yielding 21.7% of cells expressing HLA-E and 50.1% of cells expressing dual CAR. Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115).

### Example 8. Simultaneously Knockin of GC012HL at TRAC Locus and HLA-E at B2M Locus

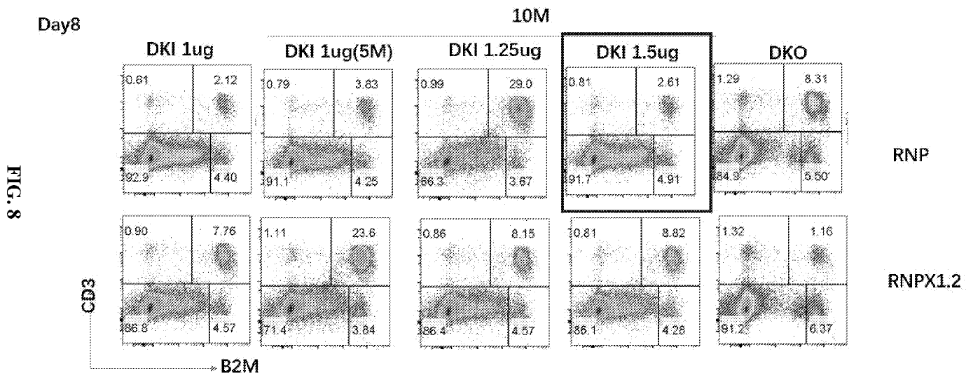

FIG. 7 illustrates knocking in of both dual CAR (Dual-CAR-T) and HLA-E at B2M locus and at TRAC locus respectively, yielding 31.8% of cells expressing HLA-E R. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). A series of concentration of chimeric polynucleotide was used, from 1 μg to 1.5 μg per 100 μl reaction per 5 or 10 million activated T cells. RNPX1.2 group was used at 1.2 times RNP as compared to RNP group. The general operation of the experiment was mixing sgRNA and Cas9 protein with PGA.

Knockin efficiency was detected on day 8 after electroporation by flow cytometry. As shown in FIG. 7, HLA-E+B2M (high) population was endogenous HLA-E, and HLA-E+B2M (low) population was exogenous, namely knockin of HLA-E. Group of cells contacted with 1.5 μg chimeric polynucleotide per ten million cells had the highest knockin efficiency at 31.8%.

### Example 9. Double Knockin had Little Effect on Knockout Efficiency

FIG. 8 illustrates knocking in of both dual CAR (Dual-CAR-T) and HLA-E at TRAC locus and at B2M locus led to knockout of TRAC at 96.57% and knockout of B2M at 92.48%. Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). A series of concentration of chimeric polynucleotide was used, from 1 μg to 1.5 μg per 100 μl reaction per 5 or 10 million activated T cells. RNPX1.2 group was used at 1.2 times RNP as compared to RNP group.

### Example 10. Chimeric Polynucleotide Comprising Telomeric Ends

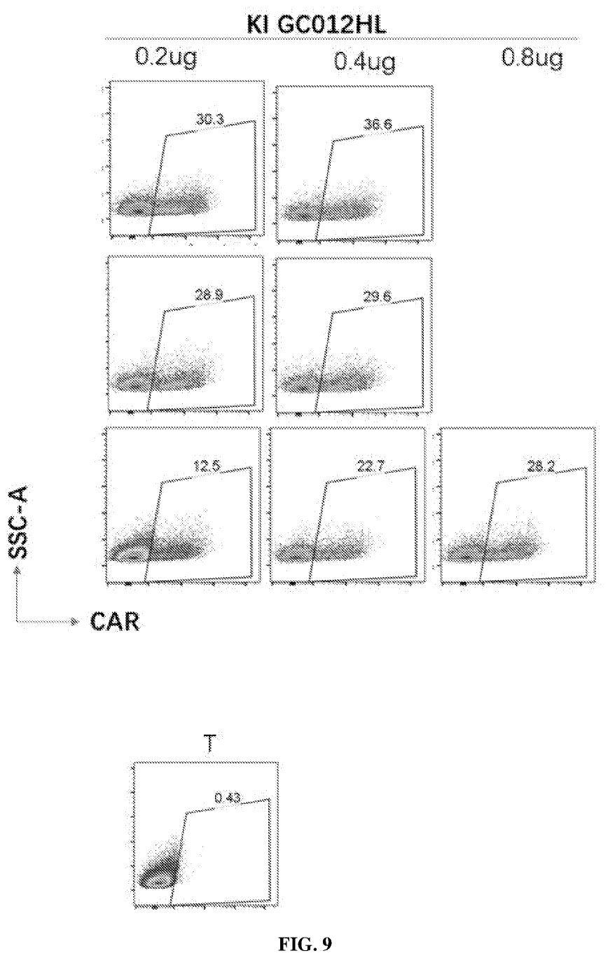

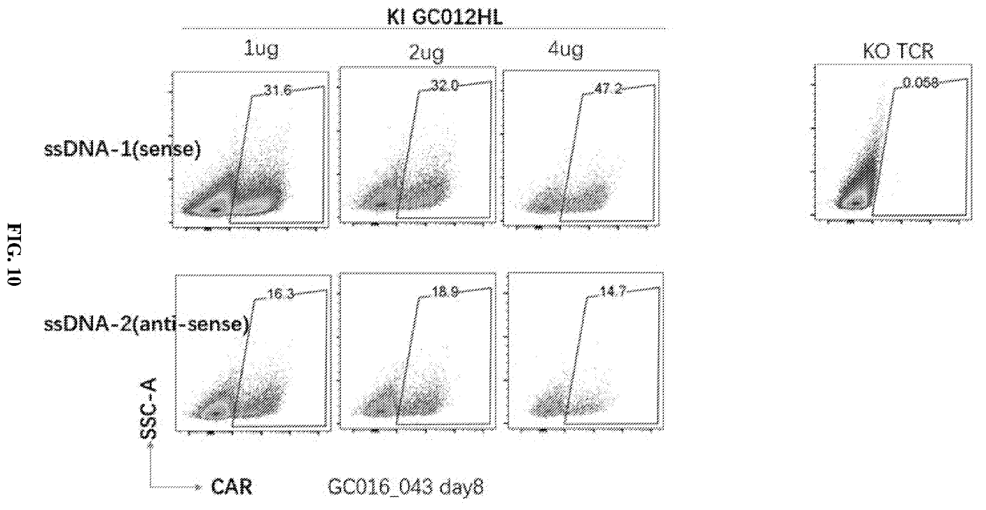

FIG. 9 illustrates knocking in of the chimeric polynucleotide, where the chimeric polynucleotide was double stranded DNA comprising the modifying moiety targeting fragment described herein and was: covalently closed on both 5′ and 3′ ends (telomeric ends, top), not covalently closed on both 5′ and 3′ ends (middle); and circular (minicircle, bottom). One million cells (in a final volume of 20 μl) were contacted with 0.20 μg, 0.40 μg, or 0.80 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). T cells served as negative control, and GC012HL was knocked into TRAC locus in this experiment. Knockin efficiency was detected on specific day after electroporation by flow cytometry. As shown in FIG. 9, the knockin efficiency of chimeric polynucleotide comprising a minicircle was similar with linear chimeric polynucleotide comprising the at least one gene modifying moiety targeting fragment without the telomeric ends, but lower than chimeric polynucleotide comprising the at least one gene modifying moiety targeting fragment with telomeric ends.

### Example 11. Knockin with Single-Stranded DNA (ssDNA) as Chimeric Polynucleotide

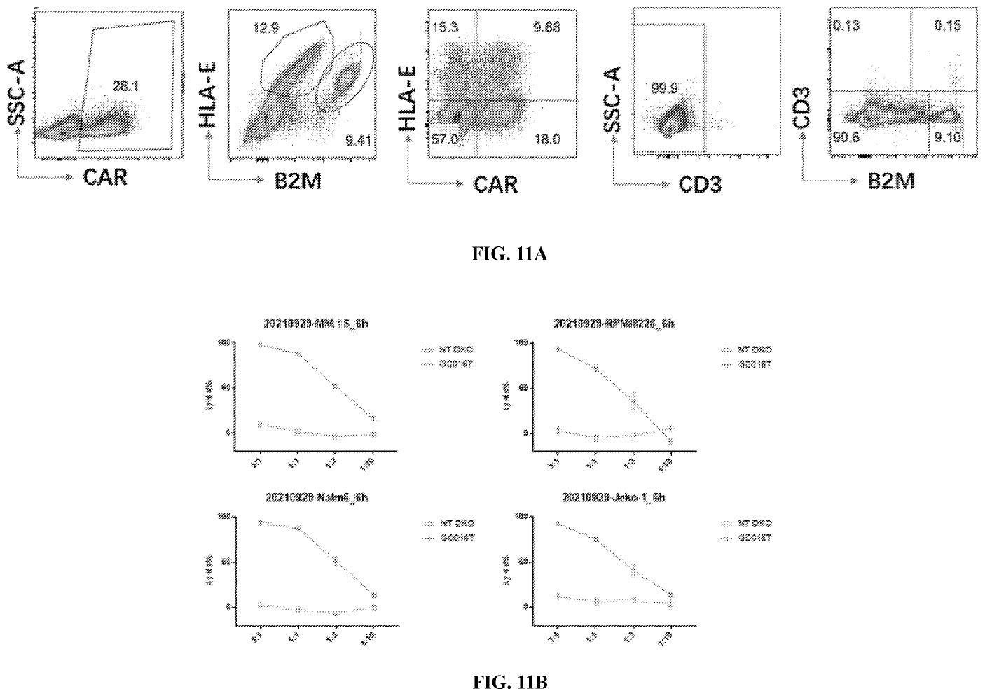

FIG. 10 illustrates knocking in of the chimeric polynucleotide, where the chimeric polynucleotide was single stranded (either sense or antisense strand). On day 8, 47.2% of the cells expressed the dual CAR (GC012HL). Ten million cells (in a final volume of 100 μl) were contacted with 1.0 μg, 1.25 μg, or 1.50 μg of the chimeric polynucleotide for this knockin experiment. The cells were incubated with Cas9/RNP at 37 degrees Celsius for 15 minutes before the addition of the various quantity of the chimeric polynucleotide for another 5 minutes of incubation at 37 degrees Celsius followed by electroporation (Lonza 4D-Nucleofector, protocol EH-115). Knockin efficiency was detected on specific day after electroporation by flow cytometry, CAR positive percentage of ssDNA sense strand knock-in group on day 8 was 47.2%.

### Example 12. Knockin of GC012HL at TRAC Locus and HLA-E at B2M Locus

FIG. 11A illustrates knocking in of both dual CAR (targeting both CD19 and BCMA) and HLA-E into cells by the system described herein. FIG. 11B illustrates the cell killing activity of the cells with the dual CAR knocked in by the system described herein. On day 8, the cells with CAR targeting CD19 or BCMA exhibited cell killing activity for killing B cell precursor leukemia cells (Nalm6, which expressed CD19) and cancer cells (MM.1S. RPMI-8226, and JeKo-1 cells, which all expressed BCMA).

### Example 13. Autologous Knockin of CAR

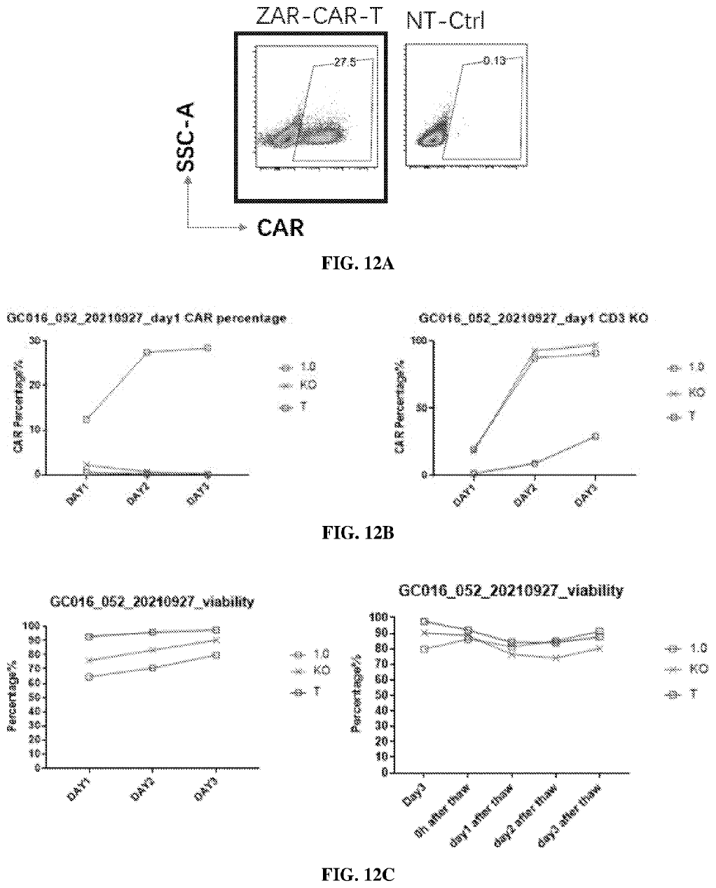

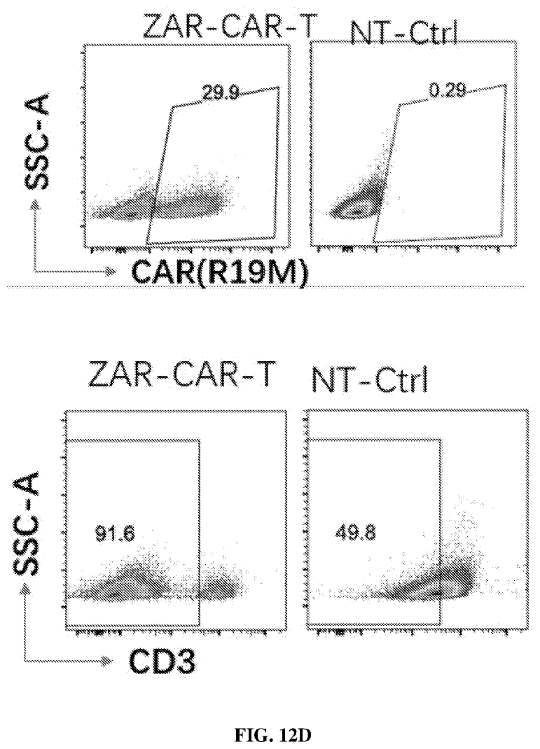

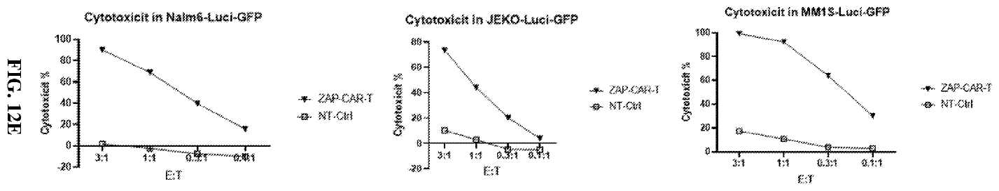

FIG. 12A-E illustrate non-viral knockin of the chimeric polynucleotide described herein for generating CAR-T cells (ZAR-CAR-T). T cell was activated for 48 hours before electroporation, and CAR positive percentage and viability were detected on day 1, 2, and 3 after electroporation. CAR expression and viability after thawing were also detected. In vitro tumor cells killing assay was performed after thawing. Data shows that ZAR-CAR-T had stable CAR expression even after thawing, and viability was up to 80% on day 3 after electroporation. ZAP-CAR-T showed adequate cell killing after six hours. FIG. 12A illustrates modified cells that were positive for exhibiting the CAR on day 2. FIG. 12B illustrates percentage of changes of modified cells that were positive for exhibiting the knockin. FIG. 12C illustrates cells viability of the modified cells with the knockin across 3 days (left) and after being thawed from cryopreservation (right). FIG. 12D illustrates viability and percentage of changes of the modified cells that were positive for exhibiting the knockin on day 1 after being thawed from cryopreservation. NT-Ctrl: no template control. Top: modified cells exhibiting CAR targeting CD19. Bottom: modified cells exhibiting CD3. FIG. 12E illustrates cell killing activity of the modified cells (ZAP-CAR-T) against cells expressing CD19 (Nalm6 cells) or BCMA (JeKo-1 and MM.1S cells).

### Example 14. Knockin Efficiency of GC012HL and HLA-E at TRAC Locus and B2M Locus

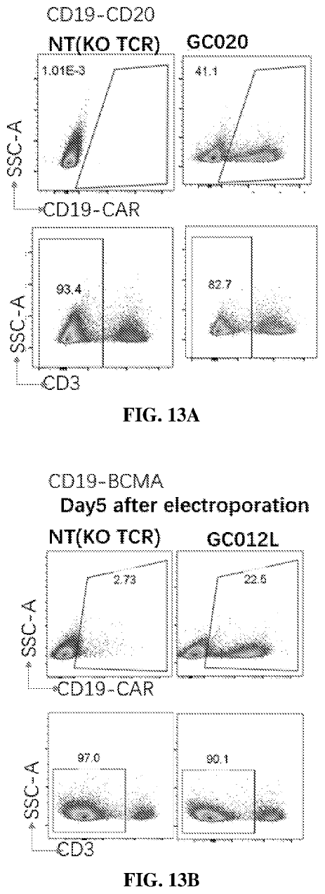

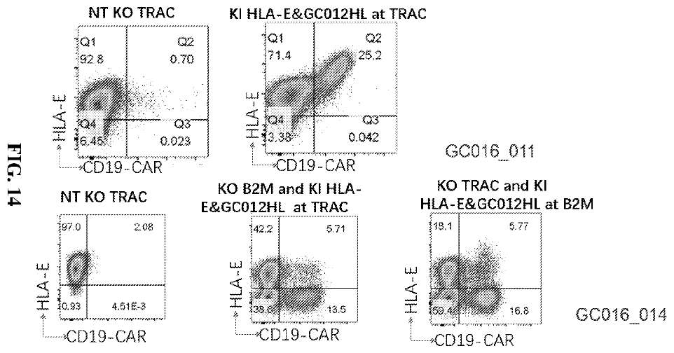

FIG. 13A illustrates knocking in of dual CAR for targeting CD19 or CD20 at a single locus of TRAC via the system described herein, 41.1% of the cells exhibited positive expression for the dual CAR for targeting CD 19 or CD20. FIG. 13B illustrates knocking in of dual CAR for targeting CD19 or BCMA at a single locus of TRAC via the system described herein, 41.1% of the cells exhibited positive expression for the dual CAR for targeting CD 19 or BCMA. FIG. 14 illustrates knocking in of chimeric polynucleotide by the system described here, where the chimeric polynucleotide knocked in at a locus such as TRAC or B2M was over five thousand bps. About 20% of the modified cells were positive for exhibiting the knockin. Flow cytometry study shows that 25.2% of the cells expressed CD19 CAR, 5.77% of the cells expressed both CD19 CAR and HLA-E.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A system comprising:
a) a guide nucleic acid;
b) a gene modifying moiety; and
c) a chimeric polynucleotide, said chimeric polynucleotide comprising: at least one expression sequence; and at least one gene modifying moiety targeting fragment, wherein said chimeric polynucleotide is circular.

2. A system comprising:
a) a guide nucleic acid;
b) a gene modifying moiety; and
c) a chimeric polynucleotide, said chimeric polynucleotide comprising: at least one expression sequence; at least one gene modifying moiety targeting fragment; and at least one covalently closed end.

3. The system of claim 2, wherein the chimeric polynucleotide is a linear DNA.

4. The system of claim 1 or 2, wherein the chimeric polynucleotide comprises double-stranded DNA (dsDNA) or singled-stranded DNA (ssDNA).

5. The system of claim 1, wherein the chimeric polynucleotide is a vector.

6. The system of claim 1, wherein the chimeric polynucleotide is a minicircle.

7. The system of claim 2, wherein the at least one covalently closed end is at 5′ end of the chimeric polynucleotide.

8. The system of claim 2, wherein the at least one covalently closed end is at 3′ end of the chimeric polynucleotide.

9. The system of claim 2, wherein the chimeric polynucleotide comprises the at least one covalently closed end 5′ end and the at least one covalently closed end at 3′ end of the chimeric polynucleotide.

10. The system of claim 1, wherein the chimeric polynucleotide is a circular single stranded DNA.

11. The system of any one of claims 2-3, wherein the chimeric polynucleotide is a linear single stranded DNA.

12. The system of any one of claims 2-3, wherein the chimeric polynucleotide is a linear single stranded DNA with modified ends.

13. The system of any one of claims 2-3 and 7-12, wherein the at least one covalently closed end comprises a telomeric end.

14. The system of any one of claims 2-3 and 7-13, wherein the chimeric polynucleotide comprises the at least one gene modifying moiety targeting fragment near 5′ end of the chimeric polynucleotide.

15. The system of any one of claims 2-3 and 7-13, wherein the chimeric polynucleotide comprises the at least one gene modifying moiety targeting fragment near 3′ end of the chimeric polynucleotide.

16. The system of any one of claims 2-3 and 7-13, wherein the chimeric polynucleotide comprises the at least one gene modifying moiety targeting fragment near 5′ end of the chimeric polynucleotide and the at least one gene modifying moiety targeting fragment near 3′ end of the chimeric polynucleotide.

17. The system of any one of the preceding claims, wherein the guide nucleic acid comprises a nucleic acid sequence that is at least 70%, at least 75%, at least 80%, at least 85%, at least 90%, at least 95%, at least 99% or more identical to a genomic sequence in a cell.

18. The system of claim 17, wherein the genomic sequence comprises a genomic locus of the cell.

19. The system of claim 18, wherein the genomic locus comprises T-cell receptor alpha chain constant (TRAC), beta-2-microglobulin (B2M), cluster of differentiation 38 (CD38), cytokine inducible SH2 containing protein (CISH), programmed cell death protein 1 (PD-1), cluster of differentiation 70 (CD70).

20. The system of claim 19, wherein the genomic locus comprises TRAC or B2M.

21. The system of any one of claims 17-20, wherein the guide nucleic acid complexes with and directs the gene modifying moiety to the genomic sequence in the cell.

22. The system of any one of the preceding claims, wherein the at least one gene modifying moiety targeting fragment comprises between about 10 nucleotide base pairs (bps) to about 100 nucleotide bps.

23. The system of any one of the preceding claims, wherein the at least one gene modifying moiety targeting fragment comprises a nucleic acid sequence that is at least 70%, at least 75%, at least 80%, at least 85%, at least 90%, at least 95%, at least 99% or more identical to the guide nucleic acid.

24. The system of any one of the preceding claims, wherein the at least one gene modifying moiety targeting fragment comprises at least one mismatch, at least two mismatches, at least three mismatches, at least four mismatches, at least five mismatches, at least six mismatches, at least seven mismatches, at least eight mismatches, at least nine mismatches, or at least ten mismatches compared to the guide nucleic acid.

25. The system of any one of the preceding claims, wherein the at least one gene modifying moiety targeting fragment complexes with the gene modifying moiety, thereby bringing the gene modifying moiety to proximity with the chimeric polynucleotide.

26. The system of any one of the preceding claims, wherein the at least one gene modifying moiety targeting fragment, when complexed with the gene modifying moiety, does not induce enzymatic activity of the gene modifying moiety.

27. The system of any one the preceding claims, wherein the gene modifying moiety comprises a Cas protein or a mRNA encoding the Cas protein.

28. The system of claim 27, wherein the gene modifying moiety comprises a Cas/RNP.

29. The system of claim 28, wherein the gene modifying moiety comprises a Cas9/RNP.

30. The system of any one of the preceding claims, wherein the at least one expression sequence encodes a chimeric receptor.

31. The system of claim 30, wherein the chimeric receptor comprises an antigen binding domain binding CD1a, CD2, CD3, CD4, CD5, CD7, CD8, CD19, CD20, CD22, CD25, CD28, CD30, CD33, CD38, CD40, CD44v6, CD47, CD52, CD56, CD57, CD58, CD70, CD79a, CD79b, CD80, CD81, CD86, CD99, CD117, CD123, CD133, CD135, CD137, CD151, CD171, CD276, BAFF-R, BCMA, B7H4, CEA, CEACM6, Claudin 18.2, CLL-1, c-Met, CS-1, CTLA-4, EGFRVIII, GPC2, GPC3, GPRC5, HER2, HER3, HER4/ErbB4, HVEM, MAGE-A, MAGE3, MSLN, MUC-1, MUC-16, NY-ESO-1, OX40, PD-1, PD-L1, PD-L2, PMSA, ROR1, TCRa, TCRb, TLR7, TLR9, VEGFR-2, WT-1, or a fragment thereof.

32. The system of claim 30 or 31, wherein the chimeric receptor comprises a transmembrane domain.

33. The system of any one of claims 30-32, wherein the chimeric receptor comprises a signaling domain.

34. The system of any one of claims 17-33, wherein the cell comprises an immune cell or a stem cell.

35. The system of claim 34, wherein the immune cell is a lymphocyte.

36. The system of claim 35, wherein the lymphocyte is a B cell.

37. The system of claim 35, wherein the lymphocyte is a T cell.

38. The system of claim 37, wherein the T cell is selected from the group consisting of: cytotoxic T cell, alpha beta T cell, a gamma delta T cell, natural killer T cell, regulatory T cell, and T helper cell.

39. The system of claim 34, wherein the immune cell comprises an ILC.

40. The system of claim 34, wherein the immune cell is derived from an iPSC.

41. The system of claim 34, wherein the immune cell is an iPSC derived T cell.

42. The system of claim 34, wherein the immune cell is an iPSC derived natural killer T cell.

43. The system of claim 34, wherein the immune cell is an iPSC derived macrophage.

44. The system of claim 34, wherein the stem cell is a hematopoietic stem cell.

45. The system of claim 34, wherein the stem cell is an iPSC.

46. The system of any one of the preceding claims, further comprising a polymer, said polymer comprises an overall anionic charge.

47. The system of claim 46, wherein said polymer comprises an anionic polynucleotide comprising poly-glutamic acid (PGA) or poly-aspartic acid (PASA).

48. A composition comprising the system of any one of the preceding claims

49. A cell line comprising the cell of any one of claims 17-48.

50. A pharmaceutical composition comprising the system of any one of claims 1-47 or the cell of claim 49.

51. The pharmaceutical composition of claim 50 comprises a unit dose form.

52. The pharmaceutical composition of claim 50 or 51, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

53. The pharmaceutical composition of any one of claims 50-52, further comprising at least one additional active agent.

54. The pharmaceutical composition of claim 53, wherein the at least one additional active agent comprises a cytokine, a growth factor, a hormone, an enzyme, a small molecule, a compound, or combinations thereof.

55. A kit comprising the system of any one of claims 1-47, the cell of claim 49, or the pharmaceutical composition of any one of claims 50-54; and a container.

56. A method comprising contacting a cell with the system of any one of claims 1-47, wherein the system knocks in the chimeric polynucleotide at a genomic sequence in the cell, thereby expressing a chimeric receptor encoded by the chimeric polynucleotide in the cell.

57. A method comprising contacting a population of cells with the system of any one of claims 1-47, wherein the system knocks in the chimeric polynucleotide at a genomic sequence in the population of cells, thereby expressing a chimeric receptor encoded by the chimeric polynucleotide in the population of cells.

58. The method of claim 57, wherein the system of any one of claims 1-47 increases knockin efficiency of the chimeric polynucleotide in the population of cells by at least 10%, at least 20%, at least 30%, at least 40%, or more compared to a Cas protein mediated knockin efficiency of the chimeric polynucleotide in absence of the at least one gene modifying moiety targeting fragment in a comparable population of cells.

59. The method of claim 57, wherein the system of any one of claims 1-47 increases an expression of the chimeric polynucleotide in the population of cells by at least 10%, at least 20%, at least 30%, at least 40%, or more compared to an expression of the chimeric polynucleotide in absence of the at least one gene modifying moiety targeting fragment knocked into a comparable population of cells by a Cas protein.

60. The method of claim 57, wherein the system of any one of claims 1-43 increases survival rate in the population of cells by at least 10%, at least 20%, at least 30%, at least 40%, or more compared to survival rate of a comparable population of cells modified by a Cas protein mediated knockin of the chimeric polynucleotide in absence of the at least one gene modifying moiety targeting fragment.

61. A method comprising contacting a cell with:
a) a first system comprising the system of any one of claims 1-47, wherein the first system comprises a first guide nucleic acid complexed with a first gene modifying moiety and a first chimeric polynucleotide comprising the at least one gene modifying moiety targeting fragment; and
b) a second system comprising the system of any one of claims 1-47, wherein the second system comprises a second guide nucleic acid complexed with a second gene modifying moiety and a second chimeric polynucleotide comprising the at least one gene modifying moiety targeting fragment,
wherein the first system introduces the first chimeric polynucleotide into a first genomic sequence in the cell and the second system introduces the second chimeric polynucleotide into a second genomic sequence in the cell.

62. A method comprising contacting a population of cells with:
a) a first system comprising the system of any one of claims 1-47, wherein the first system comprises a first guide nucleic acid complexed with a first gene modifying moiety and a first chimeric polynucleotide comprising the at least one gene modifying moiety targeting fragment; and
b) a second system comprising the system of any one of claims 1-47, wherein the second system comprises a second guide nucleic acid complexed with a second gene modifying moiety and a second chimeric polynucleotide comprising the at least one gene modifying moiety targeting fragment,
wherein the first system introduces the first chimeric polynucleotide into a first genomic sequence in the population of cells and the second system introduces the second chimeric polynucleotide into a second genomic sequence in the population of cells.

63. The method of claim 62, wherein the first system and the second system increase knockin efficiency of the first chimeric polynucleotide and the second chimeric polynucleotide in the population of cells by at least 10%, at least 20%, at least 30%, at least 40%, or more compared to a Cas protein mediated knockin efficiency of a first and a second chimeric polynucleotide in absence of the at least one gene modifying moiety targeting fragment in a comparable population of cells.

64. The method of claim 62, wherein the first system and the second system increase expressions of the first chimeric polynucleotide and the second chimeric polynucleotide in the population of cells by at least 10%, at least 20%, at least 30%, at least 40%, or more compared to expressions of the first and second chimeric polynucleotide in absence of the at least one gene modifying moiety targeting fragment knocked into a comparable population of cells by a Cas protein.

65. The method of claim 62, wherein the first system and the second system increase survival rate in the population of cells by at least 10%, at least 20%, at least 30%, at least 40%, or more compared to survival rate of a comparable population of cells modified by a Cas protein mediated knockin of a first and a second chimeric polynucleotide in absence of the at least one gene modifying moiety targeting fragment.

66. A method comprising contacting a cell with:
a) a guide nucleic acid;
b) a gene modifying moiety; and
c) a chimeric polynucleotide, said chimeric polynucleotide comprising: at least one expression sequence; and at least one gene modifying moiety targeting fragment,
wherein said chimeric polynucleotide is circular.

67. A method comprising contacting a cell with:
a) a guide nucleic acid;
b) a gene modifying moiety; and
c) a chimeric polynucleotide, said chimeric polynucleotide comprising: at least one expression sequence; at least one gene modifying moiety targeting fragment; and at least one covalently closed end.

68. The method of claim 66 or 67, wherein the chimeric polynucleotide comprises double-stranded DNA (dsDNA) or single-stranded DNA (ssDNA).

69. The method of claim 66, wherein the chimeric polynucleotide is a vector.

70. The method of claim 66, wherein the chimeric polynucleotide is a minicircle.

71. The method of claim 67, wherein the at least one covalently closed end is at 5′ end of the chimeric polynucleotide.

72. The method of claim 66, wherein the at least one covalently closed end is at 3′ end of the chimeric polynucleotide.

73. The method of claim 66, wherein the chimeric polynucleotide comprises the at least one covalently closed end 5′ end and the at least one covalently closed end at 3′ end of the chimeric polynucleotide.

74. The method of any one of claims 66 and 71-73, wherein the at least one covalently closed end comprises a telomeric end.

75. The method of any one of claims 66 and 71-74, wherein the chimeric polynucleotide comprises the at least one gene modifying moiety targeting fragment near 5′ end of the chimeric polynucleotide.

76. The method of any one of claims 66 and 71-74, wherein the chimeric polynucleotide comprises the at least one gene modifying moiety targeting fragment near 3′ end of the chimeric polynucleotide.

77. The method of any one of claims 66 and 71-74, wherein the chimeric polynucleotide comprises the at least one gene modifying moiety targeting fragment near 5′ end of the chimeric polynucleotide and the at least one gene modifying moiety targeting fragment near 3′ end of the chimeric polynucleotide.

78. The method of any one of claims 66-77, wherein the at least one gene modifying moiety targeting fragment comprises between about 10 nucleotide base pairs (bps) to about 100 nucleotide bps.

79. The method of any one of claims 66-78, wherein the at least one gene modifying moiety targeting fragment comprises a nucleic acid sequence that is at least 70%, at least 75%, at least 80%, at least 85%, at least 90%, at least 95%, at least 99% or more identical to the guide nucleic acid.

80. The method of any one of claims 66-79, wherein the at least one gene modifying moiety targeting fragment comprises at least one mismatch, at least two mismatches, at least three mismatches, at least four mismatches, at least five mismatches, at least six mismatches, at least seven mismatches, at least eight mismatches, at least nine mismatches, or at least ten mismatches compared to the guide nucleic acid.

81. The method of any one of claims 66-80, wherein the at least one gene modifying moiety targeting fragment complexes with the gene modifying moiety, thereby bringing the gene modifying moiety to proximity with the chimeric polynucleotide.

82. The method of any one of claims 66-81, wherein the at least one gene modifying moiety targeting fragment, when complexed with the gene modifying moiety, does not induce enzymatic activity of the gene modifying moiety.

83. A chimeric polynucleotide comprising:
a) at least one expression sequence; and
b) at least one gene modifying moiety targeting fragment,
wherein said chimeric polynucleotide is circular.

84. The chimeric polynucleotide of claim 83, wherein the chimeric polynucleotide is a vector.

85. The chimeric polynucleotide of claim 83, wherein the chimeric polynucleotide is a minicircle.

86. A chimeric polynucleotide comprising:
a) at least one expression sequence; a
b) at least one gene modifying moiety targeting fragment; and
c) at least one covalently closed end.

87. The chimeric polynucleotide of claim 86, wherein the at least one covalently closed end is at 5′ end of the chimeric polynucleotide.

88. The chimeric polynucleotide of claim 86, wherein the at least one covalently closed end is at 3′ end of the chimeric polynucleotide.

89. The chimeric polynucleotide of claim 86, wherein the chimeric polynucleotide comprises the at least one covalently closed end 5′ end and the at least one covalently closed end at 3′ end of the chimeric polynucleotide.

90. The chimeric polynucleotide of any one of claims 86-89, wherein the at least one covalently closed end comprises a telomeric end.

91. The chimeric polynucleotide of any one of claims 83-90, wherein the chimeric polynucleotide comprises double-stranded DNA (dsDNA) or single stranded DNA (ssDNA).

92. The chimeric polynucleotide of any one of claims 83-91, wherein the chimeric polynucleotide comprises the at least one gene modifying moiety targeting fragment near 5′ end of the chimeric polynucleotide.

93. The chimeric polynucleotide of any one of claims 80-91, wherein the chimeric polynucleotide comprises the at least one gene modifying moiety targeting fragment near 3′ end of the chimeric polynucleotide.

94. The chimeric polynucleotide of any one of claims 83-91, wherein the chimeric polynucleotide comprises the at least one gene modifying moiety targeting fragment near 5′ end of the chimeric polynucleotide and the at least one gene modifying moiety targeting fragment near 3′ end of the chimeric polynucleotide.

95. The chimeric polynucleotide of any one of claims 83-94, wherein the at least one gene modifying moiety targeting fragment comprises between about 10 nucleotide base pairs (bps) to about 100 nucleotide bps.

96. The chimeric polynucleotide of any one of claims 83-95, wherein the at least one gene modifying moiety targeting fragment complexes with the gene modifying moiety, thereby bringing the gene modifying moiety to proximity with the chimeric polynucleotide.

97. The chimeric polynucleotide of any one of claims 83-96, wherein the at least one gene modifying moiety targeting fragment, when complexed with the gene modifying moiety, does not induce enzymatic activity of the gene modifying moiety.
